SEQUENCE LISTING (310 sequences)

SEQ ID NO: 1 - protein, 9 aa, Bovine viral diarrhea virus
FQRGVNRSL

SEQ ID NO: 2 - protein, 9 aa, Bovine viral diarrhea virus
VQYTARGQL

SEQ ID NO: 3 - protein, 9 aa, Bovine viral diarrhea virus
LTIPNWRPL

SEQ ID NO: 4 - protein, 9 aa, Bovine viral diarrhea virus
VASLFISAL

SEQ ID NO: 5 - protein, 9 aa, Bovine viral diarrhea virus
YILDLIYSL

SEQ ID NO: 6 - protein, 9 aa, Bovine viral diarrhea virus
LLMYSWNPL

SEQ ID NO: 7 - protein, 9 aa, Bovine viral diarrhea virus
GEYQYWFDL

SEQ ID NO: 8 - protein, 9 aa, Bovine viral diarrhea virus
REMNYDWSL

SEQ ID NO: 9 - protein, 9 aa, Bovine viral diarrhea virus
TAFFGVMPR

SEQ ID NO: 10 - protein, 9 aa, Bovine viral diarrhea virus
SALATYTYK

SEQ ID NO: 11 - protein, 9 aa, Bovine viral diarrhea virus
YLPYATSAL

SEQ ID NO: 12 - protein, 9 aa, Bovine viral diarrhea virus
YMAGRDTAV

SEQ ID NO: 13 - protein, 9 aa, Bovine viral diarrhea virus
KQMSLTPLF

SEQ ID NO: 14 - protein, 9 aa, Bovine viral diarrhea virus
AMVEYSYIF

SEQ ID NO: 15 - protein, 9 aa, Bovine viral diarrhea virus
FAPETASVL

SEQ ID NO: 16 - protein, 9 aa, Bovine viral diarrhea virus
QQYMLKGEY

SEQ ID NO: 17 - protein, 9 aa, Bovine viral diarrhea virus
YQYWFDLEI

SEQ ID NO: 18 - protein, 9 aa, Bovine viral diarrhea virus
YMAYLTLDF

SEQ ID NO: 19 - protein, 9 aa, Bovine viral diarrhea virus
YMLKGEYQY

SEQ ID NO: 20 - protein, 9 aa, Bovine viral diarrhea virus
NMMDKLTAF

SEQ ID NO: 21 - protein, 9 aa, Bovine viral diarrhea virus
YQDYKGPVY

SEQ ID NO: 22 - protein, 9 aa, Bovine viral diarrhea virus
LMISYVTDY

SEQ ID NO: 23 - protein, 9 aa, Bovine viral diarrhea virus
AENALLIAL

SEQ ID NO: 24 - protein, 9 aa, Bovine viral diarrhea virus
YEMKALRNV

SEQ ID NO: 25 - protein, 9 aa, Bovine viral diarrhea virus
YEYSDGLQL

SEQ ID NO: 26 - protein, 9 aa, Bovine viral diarrhea virus
WQMVYMAYL

SEQ ID NO: 27 - protein, 9 aa, Bovine viral diarrhea virus
SQFLDIAGL

SEQ ID NO: 28 - protein, 9 aa, Bovine viral diarrhea virus
RTYKRVRPF

SEQ ID NO: 29 - protein, 9 aa, Bovine viral diarrhea virus
YKRWIQCVL

SEQ ID NO: 30 - protein, 9 aa, Bovine viral diarrhea virus
RDYFAESLL

SEQ ID NO: 31 - protein, 9 aa, Bovine viral diarrhea virus
AQFGAGEIV

SEQ ID NO: 32 - protein, 9 aa, Bovine viral diarrhea virus
SEVLLLSVV

SEQ ID NO: 33 - protein, 9 aa, Bovine viral diarrhea virus
LEQEVQVEI

SEQ ID NO: 34 - protein, 9 aa, Bovine viral diarrhea virus
YETATVLVF

SEQ ID NO: 35 - protein, 9 aa, Bovine viral diarrhea virus
QEYSGFVQY

SEQ ID NO: 36 - protein, 9 aa, Bovine viral diarrhea virus
RQLGILGKK

SEQ ID NO: 37 - protein, 9 aa, Bovine viral diarrhea virus
SEQKRTLLM

SEQ ID NO: 38 - protein, 9 aa, Bovine viral diarrhea virus
REHNKWILK

SEQ ID NO: 39 - protein, 9 aa, Bovine viral diarrhea virus
AMAVLTLTL

SEQ ID NO: 40 - protein, 9 aa, Bovine viral diarrhea virus
ALRDFNPEL

SEQ ID NO: 41 - protein, 9 aa, Bovine viral diarrhea virus
RQAAVDLVV

SEQ ID NO: 42 - protein, 9 aa, Bovine viral diarrhea virus
LQLQTRTSL

SEQ ID NO: 43 - protein, 9 aa, Bovine viral diarrhea virus
IMFEAFELL

SEQ ID NO: 44 - protein, 9 aa, Bovine viral diarrhea virus
KAVAFSFLL

SEQ ID NO: 45 - protein, 9 aa, Bovine viral diarrhea virus
FEEASMCEI

SEQ ID NO: 46 - protein, 9 aa, Bovine viral diarrhea virus
FEIAVSDVL

SEQ ID NO: 47 - protein, 9 aa, Bovine viral diarrhea virus
YAASPYCEV

SEQ ID NO: 48 - protein, 9 aa, Bovine viral diarrhea virus
TAATTTAFL

SEQ ID NO: 49 - protein, 9 aa, Bovine viral diarrhea virus
WPYETATVL

SEQ ID NO: 50 - protein, 9 aa, Bovine viral diarrhea virus
IPNWRPLTF

SEQ ID NO: 51 - protein, 9 aa, Bovine viral diarrhea virus
LAALHTRAL

SEQ ID NO: 52 - protein, 9 aa, Bovine viral diarrhea virus
NPLVRRICL

SEQ ID NO: 53 - protein, 9 aa, Bovine viral diarrhea virus
ITYASYGYF

SEQ ID NO: 54 - protein, 9 aa, Bovine viral diarrhea virus
YTMKLSSWF

SEQ ID NO: 55 - protein, 9 aa, Bovine viral diarrhea virus
SVIQDTAHY

SEQ ID NO: 56 - protein, 9 aa, Bovine viral diarrhea virus
GSVWNLGKY

SEQ ID NO: 57 - protein, 9 aa, Bovine viral diarrhea virus
SVYQYMRLK

SEQ ID NO: 58 - protein, 9 aa, Bovine viral diarrhea virus
STQTTYYYK

SEQ ID NO: 59 - protein, 9 aa, Bovine viral diarrhea virus
WTAATTTAF

SEQ ID NO: 60 - protein, 9 aa, Bovine viral diarrhea virus
NSMLNVLTM

SEQ ID NO: 61 - protein, 9 aa, Bovine viral diarrhea virus
GPVYHRAPL

SEQ ID NO: 62 - protein, 9 aa, Bovine viral diarrhea virus
YAIAKNDEI

SEQ ID NO: 63 - protein, 9 aa, Bovine viral diarrhea virus
SVMLGVGAI

SEQ ID NO: 64 - protein, 9 aa, Bovine viral diarrhea virus
YYDDNLNEI

SEQ ID NO: 65 - protein, 9 aa, Bovine viral diarrhea virus
FVNEDIGTI

SEQ ID NO: 66 - protein, 9 aa, Bovine viral diarrhea virus
ARRVKIHPY

SEQ ID NO: 67 - protein, 9 aa, Bovine viral diarrhea virus
LRRLRVLLM

SEQ ID NO: 68 - protein, 9 aa, Bovine viral diarrhea virus
DTYENYSFL

SEQ ID NO: 69 - protein, 9 aa, Bovine viral diarrhea virus
VMSRVIAAL

SEQ ID NO: 70 - protein, 9 aa, Bovine viral diarrhea virus
GHMASAYQL

SEQ ID NO: 71 - protein, 9 aa, Bovine viral diarrhea virus
YHIIVMHPL

SEQ ID NO: 72 - protein, 9 aa, Bovine viral diarrhea virus
QLFLRNLPI

SEQ ID NO: 73 - protein, 9 aa, Bovine viral diarrhea virus
KLANLNLSL

SEQ ID NO: 74 - protein, 9 aa, Bovine viral diarrhea virus
APVRFPTAL

SEQ ID NO: 75 - protein, 9 aa, Bovine viral diarrhea virus
YIPDKGYTL

SEQ ID NO: 76 - protein, 9 aa, Bovine viral diarrhea virus
VILSTTIYK

SEQ ID NO: 77 - protein, 9 aa, Bovine viral diarrhea virus
ATVTTWLAY

SEQ ID NO: 78 - protein, 9 aa, Bovine viral diarrhea virus
ISALATYTY

SEQ ID NO: 79 - protein, 9 aa, Bovine viral diarrhea virus
VAFSFLLMY

SEQ ID NO: 80 - protein, 9 aa, Bovine viral diarrhea virus
KVLKWVHNK

SEQ ID NO: 81 - protein, 9 aa, Bovine viral diarrhea virus
KTARNINLY

SEQ ID NO: 82 - protein, 9 aa, Bovine viral diarrhea virus
ISSKWQMVY

SEQ ID NO: 83 - protein, 9 aa, Bovine viral diarrhea virus
ISSKTGHLY

SEQ ID NO: 84 - protein, 9 aa, Bovine viral diarrhea virus
KSWLGGLDY

SEQ ID NO: 85 - protein, 9 aa, Bovine viral diarrhea virus
ITLATGAGK

SEQ ID NO: 86 - protein, 9 aa, Bovine viral diarrhea virus
FGAYAASPY

SEQ ID NO: 87 - protein, 9 aa, Bovine viral diarrhea virus
KGYNSGYYY

SEQ ID NO: 88 - protein, 9 aa, Bovine viral diarrhea virus
KSKTWFGAY

SEQ ID NO: 89 - protein, 9 aa, Bovine viral diarrhea virus
RYYETAIPK

SEQ ID NO: 90 - protein, 9 aa, Bovine viral diarrhea virus
SRDERPFVL

SEQ ID NO: 91 - protein, 9 aa, Bovine viral diarrhea virus
MLNVLTMMY

SEQ ID NO: 92 - protein, 9 aa, Bovine viral diarrhea virus
YTARGQLFL

SEQ ID NO: 93 - protein, 9 aa, Bovine viral diarrhea virus
SSAENALLI

SEQ ID NO: 94 - protein, 9 aa, Bovine viral diarrhea virus
YLKPGPLFY

SEQ ID NO: 95 - protein, 9 aa, Bovine viral diarrhea virus
KVVEPALAY

SEQ ID NO: 96 - protein, 9 aa, Bovine viral diarrhea virus
ETASVLYLV

SEQ ID NO: 97 - protein, 9 aa, Bovine viral diarrhea virus
WADFLTLIL

SEQ ID NO: 98 - protein, 9 aa, Bovine viral diarrhea virus
RVIAALIEL

SEQ ID NO: 99 - protein, 9 aa, Bovine viral diarrhea virus
ALFEAVQTI

SEQ ID NO: 100 - protein, 9 aa, Bovine viral diarrhea virus
YFEPRDNYF

SEQ ID NO: 101 - protein, 9 aa, Bovine viral diarrhea virus
YGMPKVVTI

SEQ ID NO: 102 - protein, 9 aa, Bovine viral diarrhea virus
VVTYFLLLY

SEQ ID NO: 103 - protein, 9 aa, Bovine viral diarrhea virus
YSYIFLDEY

SEQ ID NO: 104 - protein, 9 aa, Bovine viral diarrhea virus
KIMGAISDY

SEQ ID NO: 105 - protein, 9 aa, Bovine viral diarrhea virus
IAYEKAVAF

SEQ ID NO: 106 - protein, 9 aa, Bovine viral diarrhea virus
VTGSDSKLY

SEQ ID NO: 107 - protein, 9 aa, Bovine viral diarrhea virus
VTASGTPAF

SEQ ID NO: 108 - protein, 9 aa, Bovine viral diarrhea virus
ATTVVRTYK

SEQ ID NO: 109 - protein, 9 aa, Bovine viral diarrhea virus
TSMNRGDFK

SEQ ID NO: 110 - protein, 9 aa, Bovine viral diarrhea virus
KGPVSGIYL

SEQ ID NO: 111 - protein, 9 aa, Bovine viral diarrhea virus
FGPGVDAAM

SEQ ID NO: 112 - protein, 9 aa, Bovine viral diarrhea virus
WRPLTFILL

SEQ ID NO: 113 - protein, 9 aa, Bovine viral diarrhea virus
YSFLNARKL

SEQ ID NO: 114 - protein, 9 aa, Bovine viral diarrhea virus
LLPLIRATL

SEQ ID NO: 115 - protein, 9 aa, Bovine viral diarrhea virus
ATPEQLAVI

SEQ ID NO: 116 - protein, 9 aa, Bovine viral diarrhea virus
VTIIRACTL

SEQ ID NO: 117 - protein, 9 aa, Bovine viral diarrhea virus
FGYVGYQAL

SEQ ID NO: 118 - protein, 9 aa, Bovine viral diarrhea virus
YNIEPWILL

SEQ ID NO: 119 - protein, 9 aa, Bovine viral diarrhea virus
DNYFQQYML

SEQ ID NO: 120 - protein, 9 aa, Bovine viral diarrhea virus
MVYMAYLTL

SEQ ID NO: 121 - protein, 9 aa, Bovine viral diarrhea virus
TTATVRELL

SEQ ID NO: 122 - protein, 9 aa, Bovine viral diarrhea virus
ENALLIALF

SEQ ID NO: 123 - protein, 9 aa, Bovine viral diarrhea virus
VTTWLAYTF

SEQ ID NO: 124 - protein, 9 aa, Bovine viral diarrhea virus
TPSDERIRL

SEQ ID NO: 125 - protein, 9 aa, Bovine viral diarrhea virus
HPYEAYLKL

SEQ ID NO: 126 - protein, 9 aa, Bovine viral diarrhea virus
RGKFNTTLL

SEQ ID NO: 127 - protein, 9 aa, Bovine viral diarrhea virus
KGWSGLPIF

SEQ ID NO: 128 - protein, 9 aa, Bovine viral diarrhea virus
HGWCNWYNI

SEQ ID NO: 129 - protein, 9 aa, Bovine viral diarrhea virus
AGVFLIRSL

SEQ ID NO: 130 - protein, 9 aa, Bovine viral diarrhea virus
TYFLLLYLL

SEQ ID NO: 131 - protein, 9 aa, Bovine viral diarrhea virus
AGNSMLNVL

SEQ ID NO: 132 - protein, 9 aa, Bovine viral diarrhea virus
IGPLGATGL

SEQ ID NO: 133 - protein, 9 aa, Bovine viral diarrhea virus
CTFNYTRTL

SEQ ID NO: 134 - protein, 9 aa, Bovine viral diarrhea virus
DSIEVVTYF

SEQ ID NO: 135 - protein, 9 aa, Bovine viral diarrhea virus
DSKLYHIYV

SEQ ID NO: 136 - protein, 9 aa, Bovine viral diarrhea virus
RGDFKQITL

SEQ ID NO: 137 - protein, 9 aa, Bovine viral diarrhea virus
LGPIVNLLL

SEQ ID NO: 138 - protein, 9 aa, Bovine viral diarrhea virus
MTATPAGSV

SEQ ID NO: 139 - protein, 9 aa, Bovine viral diarrhea virus
NSYEVQVPV

SEQ ID NO: 140 - protein, 9 aa, Bovine viral diarrhea virus
ESGEGVYLF

SEQ ID NO: 141 - protein, 9 aa, Bovine viral diarrhea virus
LTLDFMYYM

SEQ ID NO: 142 - protein, 9 aa, Bovine viral diarrhea virus
EGRRFVASL

SEQ ID NO: 143 - protein, 9 aa, Bovine viral diarrhea virus
RGLETGWAY

SEQ ID NO: 144 - protein, 9 aa, Bovine viral diarrhea virus
IGNPLRLIY

SEQ ID NO: 145 - protein, 9 aa, Bovine viral diarrhea virus
TTTAFLVCL

SEQ ID NO: 146 - protein, 9 aa, Bovine viral diarrhea virus
VSVGISVML

SEQ ID NO: 147 - protein, 9 aa, Bovine viral diarrhea virus
TTLLNGPAF

SEQ ID NO: 148 - protein, 9 aa, Bovine viral diarrhea virus
DTKSFHEAI

SEQ ID NO: 149 - protein, 9 aa, Bovine viral diarrhea virus
SLTPLFEEL

SEQ ID NO: 150 - protein, 9 aa, Bovine viral diarrhea virus
KIHPYEAYL

SEQ ID NO: 151 - protein, 9 aa, Bovine viral diarrhea virus
KNFSFAGIL

SEQ ID NO: 152 - protein, 9 aa, Bovine viral diarrhea virus
KSFNRVARI

SEQ ID NO: 153 - protein, 9 aa, Bovine viral diarrhea virus
YHRAPLELF

SEQ ID NO: 154 - protein, 9 aa, Bovine viral diarrhea virus
LLAWAILAL

SEQ ID NO: 155 - protein, 9 aa, Bovine viral diarrhea virus
KLMSGIQTV

SEQ ID NO: 156 - protein, 9 aa, Bovine viral diarrhea virus
RRFVASLFI

SEQ ID NO: 157 - protein, 9 aa, Bovine viral diarrhea virus
KMLLATDKW

SEQ ID NO: 158 - protein, 9 aa, Bovine viral diarrhea virus
IYLKPGPLF

SEQ ID NO: 159 - protein, 9 aa, Bovine viral diarrhea virus
YEKAVAFSF

SEQ ID NO: 160 - protein, 9 aa, Bovine viral diarrhea virus
ALLGGRYVL

SEQ ID NO: 161 - protein, 9 aa, Bovine viral diarrhea virus
KLLEIFHTI

SEQ ID NO: 162 - protein, 9 aa, Bovine viral diarrhea virus
GTAKLTTWL

SEQ ID NO: 163 - protein, 9 aa, Bovine viral diarrhea virus
LAQGNWEPL

SEQ ID NO: 164 - protein, 9 aa, Bovine viral diarrhea virus
YLERVDLSF

SEQ ID NO: 165 - protein, 9 aa, Bovine viral diarrhea virus
WSDNTSSYM

SEQ ID NO: 166 - protein, 9 aa, Bovine viral diarrhea virus
VIPGSVWNL

SEQ ID NO: 167 - protein, 9 aa, Bovine viral diarrhea virus
MMDKLTAFF

SEQ ID NO: 168 - protein, 9 aa, Bovine viral diarrhea virus
YMRLKHPSI

SEQ ID NO: 169 - protein, 9 aa, Bovine viral diarrhea virus
LLRRLRVLL

SEQ ID NO: 170 - protein, 9 aa, Bovine viral diarrhea virus
VQKFINSLI

SEQ ID NO: 171 - protein, 9 aa, Bovine viral diarrhea virus
ALRNVSGSL

SEQ ID NO: 172 - protein, 9 aa, Bovine viral diarrhea virus
MEILSQNPV

SEQ ID NO: 173 - protein, 9 aa, Bovine viral diarrhea virus
IEFCSHTPV

SEQ ID NO: 174 - protein, 9 aa, Bovine viral diarrhea virus
KEHDCTSVI

SEQ ID NO: 175 - protein, 9 aa, Bovine viral diarrhea virus
AESLLVIVV

SEQ ID NO: 176 - protein, 9 aa, Bovine viral diarrhea virus
LMNKTQANL

SEQ ID NO: 177 - protein, 9 aa, Bovine viral diarrhea virus
ALSKRHVPM

SEQ ID NO: 178 - protein, 9 aa, Bovine viral diarrhea virus
AMDDKLGPM

SEQ ID NO: 179 - protein, 9 aa, Bovine viral diarrhea virus
GLWGTHTAL

SEQ ID NO: 180 - protein, 9 aa, Bovine viral diarrhea virus
GEDLYDCAL

SEQ ID NO: 181 - protein, 9 aa, Bovine viral diarrhea virus
REALEALSL

SEQ ID NO: 182 - protein, 9 aa, Bovine viral diarrhea virus
LTPLFEELL

SEQ ID NO: 183 - protein, 9 aa, Bovine viral diarrhea virus
GEIVMMGNL

SEQ ID NO: 184 - protein, 9 aa, Bovine viral diarrhea virus
SEKHLVEQL

SEQ ID NO: 185 - protein, 9 aa, Bovine viral diarrhea virus
YELVKLYYL

SEQ ID NO: 186 - protein, 9 aa, Bovine viral diarrhea virus
SQNPVSVGI

SEQ ID NO: 187 - protein, 9 aa, Bovine viral diarrhea virus
ITGAQGFPY

SEQ ID NO: 188 - protein, 9 aa, Bovine viral diarrhea virus
ALIELNWTM

SEQ ID NO: 189 - protein, 9 aa, Bovine viral diarrhea virus
RETRYLAAL

SEQ ID NO: 190 - protein, 9 aa, Bovine viral diarrhea virus
GVFLIRSLK

SEQ ID NO: 191 - protein, 9 aa, Bovine viral diarrhea virus
VNYRVTKYY

SEQ ID NO: 192 - protein, 9 aa, Bovine viral diarrhea virus
AMFQRGVNR

SEQ ID NO: 193 - protein, 9 aa, Bovine viral diarrhea virus
LSSAENALL

SEQ ID NO: 194 - protein, 9 aa, Bovine viral diarrhea virus
ALRYVAGPI

SEQ ID NO: 195 - protein, 9 aa, Bovine viral diarrhea virus
GIYLKPGPL

SEQ ID NO: 196 - protein, 9 aa, Bovine viral diarrhea virus
GENITQWNL

SEQ ID NO: 197 - protein, 9 aa, Bovine viral diarrhea virus
RECAVTCRY

SEQ ID NO: 198 - protein, 9 aa, Bovine viral diarrhea virus
GRHKRVLVL

SEQ ID NO: 199 - protein, 9 aa, Bovine viral diarrhea virus
ILLQGAPVL

SEQ ID NO: 200 - protein, 9 aa, Bovine viral diarrhea virus
ASYGYFCQM

SEQ ID NO: 201 - protein, 39 aa, Bovine parainfluenza virus 3
MEDNVQNNQIMDSWEERSGDKSSDISSALDIIEFILSTD

SEQ ID NO: 202 - protein, 39 aa, Bovine parainfluenza virus 3
MEDNAQNNQIMDSWEERSGDKSSDISSALDIIEFILSTD

SEQ ID NO: 203 - protein, 39 aa, Bovine parainfluenza virus 3; identical to SEQ ID NO: 201

SEQ ID NO: 204 - protein, 39 aa, Bovine parainfluenza virus 3; identical to SEQ ID NO: 201

SEQ ID NO: 205 - protein, 39 aa, Bovine parainfluenza virus 3; identical to SEQ ID NO: 201

SEQ ID NO: 206 - protein, 39 aa, Bovine parainfluenza virus 3; identical to SEQ ID NO: 201

SEQ ID NO: 207 - protein, 39 aa, Bovine parainfluenza virus 3
NQEAIQRRNRRGSSSDSRTETMVIRRITGGSSDPDNGTE

SEQ ID NO: 208 - protein, 39 aa, Bovine parainfluenza virus 3
NQEAIQGRNRRGSSSDSRTETLVIRRITGGSSDPDNGTE

SEQ ID NO: 209 - protein, 39 aa, Bovine parainfluenza virus 3
NQEAIQGRNRRGSSSDSRTETMVIRRITGGSSDPDNGTE

SEQ ID NO: 210 - protein, 39 aa, Bovine parainfluenza virus 3; identical to SEQ ID NO: 209

SEQ ID NO: 211 - protein, 39 aa, Bovine parainfluenza virus 3; identical to SEQ ID NO: 209

SEQ ID NO: 212 - protein, 39 aa, Bovine parainfluenza virus 3; identical to SEQ ID NO: 209

SEQ ID NO: 213 - protein, 39 aa, Bovine parainfluenza virus 3
RHTSLVTTATPDDEEELISKNKRSKRHQLTNQRDNKEIK

SEQ ID NO: 214 - protein, 39 aa, Bovine parainfluenza virus 3
RHTSLVTTATPDDEEELILKNKRSKRHQLTNQRDNKEIK

SEQ ID NO: 215 - protein, 39 aa, Bovine parainfluenza virus 3; identical to SEQ ID NO: 213

SEQ ID NO: 216 - protein, 39 aa, Bovine parainfluenza virus 3; identical to SEQ ID NO: 213

SEQ ID NO: 217 - protein, 39 aa, Bovine parainfluenza virus 3; identical to SEQ ID NO: 213

SEQ ID NO: 218 - protein, 39 aa, Bovine parainfluenza virus 3; identical to SEQ ID NO: 213

SEQ ID NO: 219 - protein, 39 aa, Bovine parainfluenza virus 3
ILNNKSSNREEQTVRNPQRSAHGQKQTMVSDRSAPEQQV

SEQ ID NO: 220 - protein, 39 aa, Bovine parainfluenza virus 3
ILNNKSNNREEQTVRNPQRSAYGQKQTMVSDRSAPEQPV

SEQ ID NO: 221 - protein, 39 aa, Bovine parainfluenza virus 3
ILNNKSSNREEQTVRNPQRSAYGQKQTMVSDRSAPEQQV

SEQ ID NO: 222 - protein, 39 aa, Bovine parainfluenza virus 3; identical to SEQ ID NO: 219

SEQ ID NO: 223 - protein, 39 aa, Bovine parainfluenza virus 3; identical to SEQ ID NO: 219

SEQ ID NO: 224 - protein, 39 aa, Bovine parainfluenza virus 3; identical to SEQ ID NO: 219

SEQ ID NO: 225 - DNA, 28 bp, Bovine parainfluenza virus 3
acttcaaaggatcgaagaggaaccagag

SEQ ID NO: 226 - DNA, 28 bp, Bovine parainfluenza virus 3
acttcaaaggatcgaagaggagccagag

SEQ ID NO: 227 - DNA, 28 bp, Bovine parainfluenza virus 3
acttcaaaggatcgaagaggaaccagag

SEQ ID NO: 228 - DNA, 28 bp, Bovine parainfluenza virus 3
acttcaaaggatcgaagaggaaccagag

SEQ ID NO: 229 - DNA, 28 bp, Bovine parainfluenza virus 3
acttcaaaggatcgaagaggaaccagag

SEQ ID NO: 230 - DNA, 28 bp, Bovine parainfluenza virus 3
acttcaaaggatcgaagaggaaccagag

SEQ ID NO: 231 - DNA, 28 bp, Bovine parainfluenza virus 3
catcctggaacatcctcaacaacaagag

SEQ ID NO: 232 - DNA, 28 bp, Bovine parainfluenza virus 3
catcctggaacatcctcaacaacaagaa

SEQ ID NO: 233 - DNA, 28 bp, Bovine parainfluenza virus 3
catcctggaacatcctcaacaacaagag

SEQ ID NO: 234 - DNA, 28 bp, Bovine parainfluenza virus 3
catcctggaacatcctcaacaacaagag

SEQ ID NO: 235 - DNA, 28 bp, Bovine parainfluenza virus 3
catcctggaacatcctcaacaacaagag

SEQ ID NO: 236 - DNA, 28 bp, Bovine parainfluenza virus 3
catcctggaacatcctcaacaacaagag

SEQ ID NO: 237 - DNA, 28 bp, Bovine parainfluenza virus 3
cagacagatcagctccagaacaacaagt

SEQ ID NO: 238 - DNA, 28 bp, Bovine parainfluenza virus 3
cagacagatcagctcccgaaccacaagt

SEQ ID NO: 239 - DNA, 28 bp, Bovine parainfluenza virus 3
cagacagatcagctcccgaacaacaagt

SEQ ID NO: 240 - DNA, 28 bp, Bovine parainfluenza virus 3
cagacagatcagctcccgaacaacaagt

SEQ ID NO: 241 - DNA, 28 bp, Bovine parainfluenza virus 3
cagacagatcagctcccgaacaacaagt

SEQ ID NO: 242 - DNA, 28 bp, Bovine parainfluenza virus 3
cagacagatcagctcccgaacaacaagt

SEQ ID NO: 243 - DNA, 28 bp, Bovine parainfluenza virus 3
ttacattattacagagtcttggtgtaat

SEQ ID NO: 244 - DNA, 28 bp, Bovine parainfluenza virus 3
ttacattattacagaatcttggtgtaat

SEQ ID NO: 245 - DNA, 28 bp, Bovine parainfluenza virus 3
ttacattattacagagtcttggtgtaat

SEQ ID NO: 246 - DNA, 28 bp, Bovine parainfluenza virus 3
ttacattattacagagtcttggtgtaat

SEQ ID NO: 247 - DNA, 28 bp, Bovine parainfluenza virus 3
ttacattattacagagtcttggtgtaat

SEQ ID NO: 248 - DNA, 28 bp, Bovine parainfluenza virus 3
ttacattattacagagtcttggtgtaat

SEQ ID NO: 249 - protein, 40 aa, Bovine parainfluenza virus 3
NQEAIQGRDRRGGNSDSRTETMVTRRISRSSSDPNNGAQI

SEQ ID NO: 250 - protein, 40 aa, Bovine parainfluenza virus 3
NQETIQGGNRGRSSSDSRAEIMVTRGISRSSPDPNNGTQI

SEQ ID NO: 251 - protein, 40 aa, Bovine parainfluenza virus 3
NQKTVQGGYRRGSSPDSRTETMVTRGISRSSPDPNNGTQI

SEQ ID NO: 252 - protein, 40 aa, Bovine parainfluenza virus 3
NQETIQGGNRGGSSSDSRAETMVTRGISRSSPDPNNGTQI

SEQ ID NO: 253 - protein, 40 aa, Bovine parainfluenza virus 3
NQEAIQGRDRRGGNSDSRTETMVTGRISRSSSDPNNGAQI

SEQ ID NO: 254 - protein, 40 aa, Bovine parainfluenza virus 3; identical to SEQ ID NO: 250

SEQ ID NO: 255 - protein, 40 aa, Bovine parainfluenza virus 3; identical to SEQ ID NO: 250

SEQ ID NO: 256 - protein, 41 aa, Bovine parainfluenza virus 3
NQETVQGGYRRGSSPDSRTETMVTRRISRSSPPDPNNGTQI

SEQ ID NO: 257 - protein, 40 aa, Bovine parainfluenza virus 3
NQEAIQRRNRRGSSSDSRTETMVIRRITGGSSDPDNGTEI

SEQ ID NO: 258 - protein, 40 aa, Bovine parainfluenza virus 3
NQEAIQGRNRRGSSSDSRTETLVIRRITGGSSDPDNGTEI

SEQ ID NO: 259 - protein, 40 aa, Bovine parainfluenza virus 3
NQEAIQGRNRRGSSSDSRTETMVIRRITRGSSDPDNGTEI

SEQ ID NO: 260 - protein, 40 aa, Bovine parainfluenza virus 3
NQEAIPGRNRRGSSSDSRTETMVIRRITGGSSDPDNGTEI

SEQ ID NO: 261 - protein, 40 aa, Bovine parainfluenza virus 3; identical to SEQ ID NO: 259

SEQ ID NO: 262 - protein, 40 aa, Bovine parainfluenza virus 3
NQEAIQGRNRRGSSSDSRTETMVIRRITGGSSDPDNGTEI

SEQ ID NO: 263 - protein, 40 aa, Bovine parainfluenza virus 3
GYTSIVTAATPDDEEELLMRNNRPRRHQSTPQGDNKGIKK

SEQ ID NO: 264 - protein, 41 aa, Bovine parainfluenza virus 3
GYTSIVTAATLDDEEELLMKNTRPKRYQSTPQEEDDKGIKK

SEQ ID NO: 265 - protein, 40 aa, Bovine parainfluenza virus 3
GYTSIVTAATLDDEEELLMKNNRPRKYQSTPQNSDKGIKK

SEQ ID NO: 266 - protein, 40 aa, Bovine parainfluenza virus 3
GYTSIVTAATLDDEEELLMKNTRPRRYQSTPQEDDKGIKK

SEQ ID NO: 267 - protein, 40 aa, Bovine parainfluenza virus 3; identical to SEQ ID NO: 263

SEQ ID NO: 268 - protein, 40 aa, Bovine parainfluenza virus 3
GYTSIVTAATLDDEEELLMKNTRPKRYQSTPQEDDKGIKK

SEQ ID NO: 269 - protein, 41 aa, Bovine parainfluenza virus 3
GYTSIVTAATLDDDEEELLMKNTRPKRYQSTPQEDDKGIKK

SEQ ID NO: 270 - protein, 41 aa, Bovine parainfluenza virus 3
GYTSIVTAATLLDDEEELLMKNNRPRKYQSTPQNSDKGIKK

SEQ ID NO: 271 - protein, 40 aa, Bovine parainfluenza virus 3
RHTSLVTTATPDDEEELISKNKRSKRHQLTNQRDNKEIKK

SEQ ID NO: 272 - protein, 40 aa, Bovine parainfluenza virus 3
RHTSLVTTATPDDEEELILKNKRSKRHQLTNQRDNKEIKK

SEQ ID NO: 273 - protein, 40 aa, Bovine parainfluenza virus 3
RHTSLVTTATPDDEEELLSKNKRSKRYQLTNQRDNKEIKK

SEQ ID NO: 274 - protein, 40 aa, Bovine parainfluenza virus 3
RHTSLVTTATPNVEEELTSKNKRSKRHQLTNQRDNKEIKK

SEQ ID NO: 275 - protein, 40 aa, Bovine parainfluenza virus 3
RHTSLVTTATPDDEEELLSKNKRSKRHQLTNQRDNKEIKK

SEQ ID NO: 276 - protein, 40 aa, Bovine parainfluenza virus 3
RHTSLVTTATPDDEEELFSKNKRSKRHQLTTQRDNKEIKK

SEQ ID NO: 277 - DNA, 59 bp, Bovine parainfluenza virus 3
caaaacaacagaaacaagcaaggaaaatagtggaccagctaacaaaaatcgacagtttg

SEQ ID NO: 278 - DNA, 59 bp, Bovine parainfluenza virus 3
caaaacaacagaaacaagcaaggaaaatagtggatcagctaacaaaaatcgacagtttg

SEQ ID NO: 279 - DNA, 59 bp, Bovine parainfluenza virus 3; identical to SEQ ID NO: 277

SEQ ID NO: 280 - DNA, 59 bp, Bovine parainfluenza virus 3
agatagaaatgttaatcaggagactgtacagggaggatataggagaggaagcagcccag

SEQ ID NO: 281 - DNA, 59 bp, Bovine parainfluenza virus 3
agatagaaatgttaatcaggagactatacagggaggatataggagaggaagcagctcag

SEQ ID NO: 282 - DNA, 59 bp, Bovine parainfluenza virus 3; identical to SEQ ID NO: 280

SEQ ID NO: 283 - DNA, 59 bp, Bovine parainfluenza virus 3
aatctccagaagcagcccagatcctaacaatggaacccaaatccaggaagatattgatt

SEQ ID NO: 284 - DNA, 59 bp, Bovine parainfluenza virus 3; identical to SEQ ID NO: 283

SEQ ID NO: 285 - DNA, 59 bp, Bovine parainfluenza virus 3; identical to SEQ ID NO: 283

SEQ ID NO: 286 - DNA, 59 bp, Bovine parainfluenza virus 3; identical to SEQ ID NO: 283

SEQ ID NO: 287 - DNA, 59 bp, Bovine parainfluenza virus 3
aatctccagaagcagcccagatcctaacaatggaacccaaatccaagaagatattgatt

SEQ ID NO: 288 - DNA, 59 bp, Bovine parainfluenza virus 3; identical to SEQ ID NO: 283

SEQ ID NO: 289 - DNA, 59 bp, Bovine parainfluenza virus 3
tgatgatggaagaggcctggaatctatcagtacatttgattcaggatataccagtatag

SEQ ID NO: 290 - DNA, 59 bp, Bovine parainfluenza virus 3; identical to SEQ ID NO: 289

SEQ ID NO: 291 - DNA, 59 bp, Bovine parainfluenza virus 3; identical to SEQ ID NO: 289

SEQ ID NO: 292 - DNA, 75 bp, Bovine parainfluenza virus 3
tggtatgttggatacaacaaaatcactaattcgagtagggataagcagaggaggattaacctataacttattaag

SEQ ID NO: 293 - DNA, 74 bp, Bovine parainfluenza virus 3
aggaatgctggacacaacaaaatcgttaatcgagtagggataaatcgaggagggttaacttatagtttgttaag

SEQ ID NO: 294 - DNA, 74 bp, Bovine parainfluenza virus 3; identical to SEQ ID NO: 293

SEQ ID NO: 295 - DNA, 74 bp, Bovine parainfluenza virus 3; identical to SEQ ID NO: 293

SEQ ID NO: 296 - DNA, 74 bp, Bovine parainfluenza virus 3
aaagataagcaactatgatcttgtacaatatgagacacttagtaaaactttaagactaatagtcagtgacaaga

SEQ ID NO: 297 - DNA, 75 bp, Bovine parainfluenza virus 3
aaagataagtaactatgatcttgtacaatatgagacacttagtagaaccttaagattaattgttagtgataagat

SEQ ID NO: 298 - DNA, 75 bp, Bovine parainfluenza virus 3
aaaggtaagtaactatgatcttgtacaatatgagacacttagtagaaccttaagattaattgttagtgataagat

SEQ ID NO: 299 - DNA, 75 bp, Bovine parainfluenza virus 3; identical to SEQ ID NO: 298

SEQ ID NO: 300 - DNA, 75 bp, Bovine parainfluenza virus 3
taagtatgaagatatgtgctcagtagacctagccatatcattaagacaaaaaatgtggatgcatttatcaggagg

SEQ ID NO: 301 - DNA, 75 bp, Bovine parainfluenza virus 3
aaagtatgaagacatgtgctcggtagatctagctatctcgttaagacaaaaaatgtggatgcatttatcaggagg

SEQ ID NO: 302 - DNA, 75 bp, Bovine parainfluenza virus 3; identical to SEQ ID NO: 301

SEQ ID NO: 303 - DNA, 75 bp, Bovine parainfluenza virus 3; identical to SEQ ID NO: 301

SEQ ID NO: 304 - DNA, 75 bp, Bovine parainfluenza virus 3
aagaatgataaatggacttgaaactccagatcctttagagttactgtctggagtaataataacaggatctgaaca

SEQ ID NO: 305 - DNA, 111 bp, Artificial Sequence
  Forward primer
gcgtcgactaatacgactcactatagggagaaacatctgatgagtccgtgaggacgaaacggagtctagactccgtcatgttgagtctgtttgatacattcagtgcacgca

SEQ ID NO: 306 - DNA, 38 bp, Artificial Sequence
  Reverse primer
gcaagcttttagctacttccgaatgcgctgaacaggtc

SEQ ID NO: 307 - DNA, 111 bp, Artificial Sequence
  Forward primer
gcgtcgactaatacgactcactatagggagatccatctgatgagtccgtgaggacgaaacggagtctagactccgtcatggaagacaatgttcaaaacaatcaaatcatgg

SEQ ID NO: 308 - DNA, 60 bp, Artificial Sequence
  Reverse primer
gcaagcttctattgggagctaatgtcttcattaaacatatccatcaattcagatacttct

SEQ ID NO: 309 - DNA, 101 bp, Artificial Sequence
  Forward primer
gccccgggtaatacgactcactatagggagatccatctgatgagtccgtgaggacgaaacggagtctagactccgtcatggacaccgaattcagcggtggc

SEQ ID NO: 310 - DNA, 60 bp, Artificial Sequence
  Reverse primer
gcaagcttttaatcaatatcaaattcattatcatattcataatctggatatgattggtgt

(19) United States
(12) Patent Application Publication
(10) Pub. No.: US 2025/0325645 A1
(43) Pub. Date: Oct. 23, 2025

(54) BOVINE VIRAL DIARRHEA VIRUS IMMUNOGENIC COMPOSITIONS AND METHODS OF USE THEREOF

(71) Applicant: KANSAS STATE UNIVERSITY RESEARCH FOUNDATION, Manhattan, KS (US)

(72) Inventor: Waithaka MWANGI, Manhattan, KS (US)

(21) Appl. No.: 18/547,842

(22) PCT Filed: Mar. 11, 2022
(86) PCT No.: PCT/US2022/071108

§ 371 Date: Mar. 18, 2024

Related U.S. Application Data
(60) Provisional application No. 63/200,516, filed on Mar. 11, 2021.
(60) Provisional application No. 63/201,625, filed on May 6, 2021.

(51) Int. Cl. A61K 39/12; A61K 39/00; C12N 15/86

(52) U.S. Cl. CPC A61K 39/12; C12N 15/86; A61K 2039/5256; A61K 2039/552; C12N 2760/18643; C12N 2770/24322; C12N 2770/24333

(57) ABSTRACT

The present disclosure provides an immunogenic composition and methods of treating, preventing, and reducing the duration, incidence, and/or severity of clinical signs or symptoms of BVDV infection. The immunogenic composition includes at least one bovine MHC I-binding peptide. In some forms, the immunogenic composition includes a BPI3Vc vector expressing at least an antigenic CD8+ T cell epitope derived from at least one bovine viral diarrhea virus (BVDV) antigen selected from the group consisting of N^{pro}, E^{rns}, E1, E2, NS2-3, NS4A-B, NS5A-B, and any combination thereof.

### CROSS REFERENCE TO RELATED APPLICATIONS

This application claims the priority benefit of U.S. Provisional Application Ser. Nos. 63/200,516 and 63/201,625, which are hereby incorporated by reference in their entireties.

### STATEMENT REGARDING FEDERALLY SPONSORED RESEARCH & DEVELOPMENT

The invention was made with government support under Agriculture and Food Research Initiative Competitive Grant number 2017-67015-26802 from the USDA National Institute of Food and Agriculture. The government has certain rights in the invention.

### SEQUENCE LISTING

This application contains a sequence listing in computer readable format, the teachings and content of which are hereby incorporated by reference.

### BACKGROUND OF THE INVENTION

The field of the invention relates generally to immunogenic compositions for decreasing the incidence and severity of clinical signs or symptoms caused by or associated with infection with Bovine Viral Diarrhea Virus (BVDV). BVDV is an immunosuppressive viral pathogen that triggers multifactorial Bovine Respiratory Disease (BRD) in feedlot cattle and therefore, has a huge economic impact on various aspects of cattle industry. The 12.5 kb long single-stranded RNA genome of BVDV encodes four structural antigens, capsid, E^{rns}, E1, and E2; and seven nonstructural antigens, N^{pro}, p7, NS2-3, NS4A-B, and NS5A-B. The BVDV, a Pestivirus belonging to the Flaviviridae family, is a heterogeneous pathogen that is categorized into two antigenically distinct genotypes, BVDV-1 and -2, which are further subdivided into various sub-genotypes. BVDV strains are also classified into two biotypes, cytopathic and non-cytopathic strains. The BVDV causes transient or persistent infection (PI) in cattle often making them susceptible to secondary pathogens associated with BRD which, in turn, causes increased morbidity and mortality. Thus, management of BRD prevalence through deployment of effective counter-measures is expected to benefit the cattle industry. In the United States, modified-live virus (MLV) and killed virus (KV) BVDV vaccines have been in the market for almost six decades. Although commercial BVDV vaccines are widely used as part of the BRD management strategy in the United States, BVDV remains widespread in herds. For the MLV and KV vaccines, along with the safety-related issues, diversity of BVDV strains continues to be a challenge especially, as new variants emerge in endemic areas. Therefore, what is needed is a more efficacious, broadly protective BVDV vaccine for better BRD management.

MLV and KV provide different levels of protection whereby, they mostly elicit BVDV-specific antibody and CD4⁺ T cell responses to protect cattle. Unlike KV, the MLV also induces BVDV-specific CD8⁺ T cells which is one of the key features that makes MLV more efficacious. BVDV-specific CD4⁺ and CD8⁺ T cells are also elicited in cattle during infection and in the absence of BVDV neutralizing antibody response, BVDV-specific T cell responses provide protection. Additionally, there are defined MHC-DR-restricted epitopes within E2 and NS3 that drive BVDV-specific CD4⁺ T cells. However, cytotoxic CD8⁺ T lymphocytes (CTLs) targets have not been identified in BVDV. CTLs against Classical Swine Fever Virus (CSFV), another Pestivirus from Flaviviridae family, are elicited by E2 and NS3 antigens which have been found to contain broadly reactive CD8⁺ T cell epitopes. Structural and nonstructural antigens from Flavisviruses, such as Hepatitis C Virus (HCV) and Zika Virus, have been used to develop T cell-based vaccine candidates that expand the breadth of protective cellular immunity against heterologous infections.

### BRIEF DESCRIPTION OF THE DISCLOSURE

The present disclosure overcomes the problems inherent in the art and provides broadly protective immunogenic compositions effective for decreasing the incidence of and/or severity of clinical signs or symptoms of infection with BVDV. Preferably and advantageously, the immunogenic compositions are effective against both BVDV-1 and BVDV-2 strains.

Considering the undermining effects of hypervariable neutralizing epitopes on current BVDV vaccines' efficacy, a CTL-based vaccine capable of priming potent and sustained cross-protective CD8⁺ T cells were investigated to determine if they can help overcome BVDV antigenic diversity strains. Subunit vaccines that contain E2 and NS3 antigens tend to be more efficacious than a vaccine that contains only E2 antigen, which suggests that NS3-specific T cell responses have synergistic role in providing BVDV-specific immunity. Thus, it is believed that besides E2 and NS3 antigens, inclusion of additional T cell targets, specifically CTL determinants from other structural and non-structural BVDV antigens which comprise ˜75% of BVDV polyprotein, can markedly boost protective efficacy of a CTL-based BVDV vaccine.

Recent advances in immunoinformatics, rapid genome sequencing, and the availability of prediction algorithms have revolutionized the once labor-intensive epitope discovery as putative epitopes can be identified by proteome-wide computational analysis. This approach has transformed subunit vaccine development by enabling rapid identification of T cell epitopes from emerging human pathogens. Similarly, these tools can be applied to identify novel major histocompatibility complex (MHC) class I-restricted CD8⁺ T cell epitopes from economically significant livestock pathogens. Usually, CD8⁺ T cell epitope mapping focusses on few epitopes that bind a single prevalent MHC I allele. But given the diversity among the highly polymorphic MHC I genes, wider array of MHC I alleles along with promiscuous epitopes should be considered for the investigation of CD8⁺ T cell repertoire at population level.

In this disclosure, the full-length BVDV polyprotein was screened for bovine MHC I-binding 9-mers to identify putative novel CD8⁺ T cell epitopes using NetMHCpan2.8. The top two-hundred peptides that were predicted as the strongest binders for the available bovine leukocyte antigen (BoLA) I alleles were selected for further ex vivo screening. The cross-reactivity of CD8⁺ T cells against heterologous Flaviviruses is well known and expansion of these broad spectrum responses can be achieved by multiple heterologous immunizations. Therefore, using this as an experimental model, outbred cattle were first infected with a BVDV-1b strain (CA401186a) and after recovery, the cattle were given multiple immunizations of either an irradiated heterologous BVDV-1b (TGAC) or -2a (A125) strain. Since irradiated virus retains the ability to infect host cells like the live virus, the cattle were immunized with gamma-irradiated BVDV to ensure the presentation of BVDV antigens by BoLA I for amplification of BVDV-specific CD8⁺ T cells in vivo. Purified CD8⁺ T cells from splenocytes of these BVDV hyper-immunized cattle were used to screen the predicted 9-mer peptides by IFN-γ enzyme-linked immunospot (ELISPOT) assay. As a result, novel CD8⁺ T cell epitopes were identified from BVDV structural and nonstructural antigens. Most of these bovine MHC I-binding epitopes, which recalled IFN-γ-secreting CD8⁺ T cells in BVDV-1 and -2 immunized cattle, are highly conserved across the two genotypes. These findings strongly support the hypothesis that, a contemporary vaccine that targets highly conserved BVDV-specific CD8⁺ T cell responses will confer broad protection and reduce prevalence which will potentially lead to BVDV eradication.

In some aspects, the disclosure provides an immunogenic composition comprising at least one BVDV bovine MHC I-binding peptide or epitope. In some forms, the epitope is derived from an antigenic portion of a protein expressed by BVDV. In some forms, the at least one BVDV peptide or epitope is a CD8+ T cell epitope. In some forms, the at least one BVDV peptide or epitope is derived from BVDV-1 or BVDV-2. In some forms, the at least one BVDV-1 peptide is derived from a BVDV-1a or BVDV-1b. In some forms, the at least one BVDV-2 peptide is derived from BVDV-2a. In some forms, the at least one BVDV peptide is derived from a region selected from the group consisting of N^{pro}, E^{rns}, E1, E2, NS2, NS3, NS4A, NS4B, NS5A, and NS5B. In some forms, the at least one BVDV peptide is selected from the group consisting of SEQ ID NOS. 1-200 and any combination thereof. In some forms, the at least one BVDV peptide or epitope is selected from the group consisting of SEQ ID NOS. 32, 34, 37, 38, 39, 40, 43, 45, 47, 56, 61, 63, 64, 65, 69, 81, 82, 86, 87, 88, 89, 97, 99, 100, 172, 173, 176, 177, and any combination thereof. In some preferred forms, the epitope has a sequence that has at least 85, 86, 87, 88, 89, 90, 91, 92, 93, 94, 95, 96, 97, 98, 99, 99.5, 99.6, 99.7, 99.8, 99.9, or even 100% sequence identity or sequence homology with a sequence selected from any one of SEQ ID NOS. 61, 45, 176, 88, 86, 47, 32, 56, 34, 100, 39, 97, 82, 69, 87, 177, 172, 63, 37, 99, 43, 64, 65, 81, 40, 38, 89, 173, or any combination thereof. In some forms, at least 2, 3, 4, 5, 6, 7, 8, 9, 10, 11, 12, 13, 14, 15, 16, 17, 18, 19, 20, 21, 22, 23, 24, 25, 26, 27, or 28 antigenic epitopes are used in an immunogenic composition. In some forms, when more than one antigenic epitope is used, they are individually and respectively selected from the group consisting of SEQ ID NOS. 1-200. In some preferred forms, when more than one antigenic epitope is used, they are individually and respectively selected from the group consisting of SEQ ID NOS. 61, 45, 176, 88, 86, 47, 32, 56, 34, 100, 39, 97, 82, 69, 87, 177, 172, 63, 37, 99, 43, 64, 65, 81, 40, 38, 89, and 173. In some forms, nucleic acids coding for the antigenic epitope(s) are placed into a vector for expression. In some forms, the vector with the antigenic epitope(s) is administered to a subject in need thereof as a nucleic acid-based composition. In some forms, the antigenic epitopes are expressed and combined into a subunit-based immunogenic composition. In some forms, the vector is from bovine parainfluenza (BPI). In some forms, the vector is from BPI3Vc. In some forms, the vector has a sequence having at least 90, 91, 92, 93, 94, 95, 96, 97, 98, 99, 99.5, 99.6, 99.7, 99.8, 99.9, or even 100% sequence homology or sequence identity with SEQ ID NO. 292 or a mutant BPI3Vc vector as described below. In some forms, the immunogenic compositions of the disclosure provide protection against clinical signs of infection by at least two, and preferably all three BVDV strains (BVDV-1a, 1b, and BVDV-2). In some forms, administration of the immunogenic compositions of the disclosure reduce the incidence of or the severity of at least one clinical sign of BVDV infection. In some forms, the reduction is in comparison to an animal that has not received an administration of the immunogenic composition of the disclosure. In some forms, the reduction is at least 10, 20, 30, 40, 50, 60, 70, 80, 90, 95, 96, 97, 98, 99, or even complete prevention of at least one clinical sign of BVDV infection. In some forms, the incidence or severity can be determined in a single animal or a group of animals. In some forms, administration of a composition of the disclosure results in a reduction of the likelihood that a pregnant cow will deliver a persistently-infected animal that will shed virus for its lifetime.

The term “derived from” with respect to the epitopes and sequences disclosed herein refers to the origin or basis for the peptide or coding sequence for the peptide. For example, SEQ ID NO. 61 could be derived from BVDV 1a, 1b, or 2a, or could be derived from the Npro portion of such BVDV types. In some forms, the peptide or coding sequence is recombinant, but is considered to be derived from the BVDV type or the portion.

Immunogenic compositions comprising any of the disclosed immunogenic compositions provided herewith are very effective in reducing the severity of or incidence of clinical signs associated with BVDV infection up to and including the prevention of such signs. Further, such immunogenic compositions reduce the transmissibility of BVDV.

In one aspect, the immunogenic composition or vaccine of the present disclosure further comprises at least one additional element. The at least one additional element is preferably selected from, but not limited to, pharmaceutical-acceptable-carrier(s) and/or veterinary-acceptable carrier(s), diluent(s), solvent(s), dispersion media, coating(s), adjuvant(s), one or more antigens from pathogens other than BVDV, preservatives, isotonic agent(s), adsorption delaying agent(s), protectant(s), antibacterial and/or antifungal agent(s), stabilizers, colors, flavors, and any combination(s) thereof. In preferred forms, when the immunogenic composition includes antigens from pathogens other than BVDV, the antigens are effective for reducing the severity of or the incidence of clinical signs or symptoms of sickness or disease caused by or associated with the pathogen from which it is derived. Such compositions that include a BVDV peptide and one or more antigens from a pathogen other than BVDV are referred to as combination vaccines or combination immunogenic compositions.

“Adjuvants” as used herein, can include aluminum hydroxide and aluminum phosphate, saponins e.g., Quil A, QS-21 (Cambridge Biotech Inc., Cambridge MA), GPI-0100 (Galenica Pharmaceuticals, Inc., Birmingham, AL), water-in-oil emulsion, oil-in-water emulsion, water-in-oil-in-water emulsion. The emulsion can be based in particular on light liquid paraffin oil (European Pharmacopea type); isoprenoid oil such as squalane or squalene oil resulting from theoligomerization of alkenes, in particular of isobutene or decene; esters of acids or of alcohols containing a linear alkyl group, more particularly plant oils, ethyl oleate, propylene glycol di-(caprylate/caprate), glyceryl tri-(caprylate/caprate) or propylene glycol dioleate; esters of branched fatty acids or alcohols, in particular isostearic acid esters. The oil is used in combination with emulsifiers to form the emulsion. The emulsifiers are preferably nonionic surfactants, in particular esters of sorbitan, of mannide (e.g. anhydromannitol oleate), of glycol, of polyglycerol, of propylene glycol and of oleic, isostearic, ricinoleic or hydroxystearic acid, which are optionally ethoxylated, and polyoxypropylene-polyoxyethylene copolymer blocks, in particular the Pluronic products, especially L121. See Hunter et al., The Theory and Practical Application of Adjuvants (Ed.Stewart-Tull, D. E. S.). JohnWiley and Sons, NY, pp51-94 (1995) and Todd et al., Vaccine 15:564-570 (1997).

For example, it is possible to use the SPT emulsion described on page 147 of “Vaccine Design, The Subunit and Adjuvant Approach” edited by M. Powell and M. Newman, Plenum Press, 1995, and the emulsion MF59 described on page 183 of this same book.

A further instance of an adjuvant is a compound chosen from the polymers of acrylic or methacrylic acid and the copolymers of maleic anhydride and alkenyl derivative. Advantageous adjuvant compounds are the polymers of acrylic or methacrylic acid which are cross-linked, especially with polyalkenyl ethers of sugars or polyalcohols. These compounds are known by the term carbomer (Phameuropa Vol. 8, No. 2, June 1996). Persons skilled in the art can also refer to U.S. Pat. No. 2,909,462 which describes such acrylic polymers cross-linked with a polyhydroxylated compound having at least 3 hydroxyl groups, preferably not more than 8, the hydrogen atoms of at least three hydroxyls being replaced by unsaturated aliphatic radicals having at least 2 carbon atoms. The preferred radicals are those containing from 2 to 4 carbon atoms, e.g. vinyls, allyls and other ethylenically unsaturated groups. The unsaturated radicals may themselves contain other substituents, such as methyl. The products sold under the name Carbopol; (BF Goodrich, Ohio, USA) are particularly appropriate. They are cross-linked with an allyl sucrose or with allyl pentaerythritol. Among then, there may be mentioned Carbopol 974P, 934P and 971P. Among the copolymers of maleic anhydride and alkenyl derivative, the copolymers EMA (Monsanto) which are copolymers of maleic anhydride and ethylene. The dissolution of these polymers in water leads to an acid solution that will be neutralized, preferably to physiological pH, in order to give the adjuvant solution into which the immunogenic, immunological or vaccine composition itself will be incorporated.

Further suitable adjuvants include, but are not limited to, the RIBI adjuvant system (Ribi Inc.), Block co-polymer (CytRx, Atlanta GA), SAF-M (Chiron, Emeryville CA), monophosphoryl lipid A, Avridine lipid-amine adjuvant, heat-labile enterotoxin from E. coli (recombinant or otherwise), cholera toxin, IMS 1314 or muramyl dipeptide among many others.

Preferably, the adjuvant is added in an amount of about 100 μg to about 10 mg per dose. Even more preferably, the adjuvant is added in an amount of about 100 μg to about 10 mg per dose. Even more preferably, the adjuvant is added in an amount of about 500 μg to about 5 mg per dose. Even more preferably, the adjuvant is added in an amount of about 750 μg to about 2.5 mg per dose. Most preferably, the adjuvant is added in an amount of about 1 mg per dose.

A “protectant” as used herein, refers to an anti-microbiological active agent, such as for example Gentamycin, Merthiolate, and the like. In particular, adding a protectant is most preferred for the preparation of a multi-dose composition. Those anti-microbiological active agents are added in concentrations effective to prevent the composition of interest from any microbiological contamination or for inhibition of any microbiological growth within the composition of interest.

In some preferred forms, the present disclosure contemplates immunogenic or vaccine compositions comprising from about 1 ug/ml to about 60 μg/ml of protectan, and more preferably less than about 30 μg/ml of protectant.

In some preferred forms, the composition comprises at least one component selected from the group consisting of at least one additional antigen from a pathogen other than BVDV, stabilizing agents, preservatives, antibacterial and antifungal agents, adjuvants, adsorption delaying agents, and any combination(s) thereof.

A “stabilizing agent”, as used herein, refers to an ingredient, such as for example saccharides, trehalose, mannitol, saccharose, albumin and alkali salts of ethylendiamintetracetic acid, and the like, to increase and/or maintain product shelf-life and/or to enhance stability.

Those of skill in the art will understand that the immunogenic composition herein may incorporate known injectable, physiologically acceptable, sterile solutions. For preparing a ready-to-use solution for parenteral injection or infusion, aqueous isotonic solutions, such as e.g. saline or corresponding plasma protein solutions are readily available. In addition, as noted above, the immunogenic and vaccine compositions of the present disclosure can include diluents, isotonic agents, stabilizers, or adjuvants. Diluents can include water, saline, dextrose, ethanol, glycerol, and the like. Isotonic agents can include sodium chloride, dextrose, mannitol, sorbitol, and lactose, among others. Suitable adjuvants and stabilizers, are those described above.

According to a further aspect, the immunogenic composition of the present disclosure further comprises a pharmaceutical acceptable salt, preferably a phosphate salt in physiologically acceptable concentrations. Preferably, the pH of said immunogenic composition is adjusted to a physiological pH, meaning between about 6.5 and 7.5.

According to a further aspect, the immunogenic compositions described herein can further include one or more other immunomodulatory agents such as, e. g., interleukins, interferons, or other cytokines.

According to a further aspect, the immunogenic compositions described herein can further include an immune stimulant. It is understood that any immune stimulant known to a person skilled in the art can also be used. “Immune stimulant” as used herein, means any agent or composition that can trigger a general immune response, preferably without initiating or increasing a specific immune response, for example the immune response against a specific pathogen.

In another aspect, the present disclosure provides a method for treating, preventing, reducing the duration, incidence, or severity of clinical symptoms or signs associated with BRD and/or caused by infection with BVDV. The method preferably includes the steps of administration of the immunogenic composition or vaccine of the present disclosure to an animal or human in need thereof. The dosage is preferably provided in an effective amount. Preferably, clinical symptoms in adult cattle are selected from, but not limited to, fever and especially fever of at least 105° C., lethargy, loss of appetite, reduced weight gain, abortion, ocular discharge, nasal discharge, oral lesions, diarrhea, decreasing milk production, pneumonia including calf pneumonia, reproductive disorders, increased occurrence of other diseases, and death. The losses from fetal infection include abortions; congenital defects; weak and abnormally small calves; unthrifty, persistently infected (PI) animals that shed infectious BVDV; and death among PI animals. Chronic infection may lead to signs of mucosal disease. In calves, the most commonly recognized birth defect is cerebellar hypoplasia. The signs of this are: ataxia/lack of voluntary coordination of muscle movements; tremors; wide stance; stumbling; failure to nurse; and death.

The clinical signs or symptoms are preferably reduced in duration, incidence, or severity by about 20%, 30%, 40%, 50%, 60%, 70%, 80%, 90%, 95%, or even by 100% when compared to those animals or humans not provided the immunogenic composition or vaccine of the present disclosure. Such reduction can be applied to individual animals as well as groups or herds of animals.

The method preferably includes the steps of administration of the immunogenic composition or vaccine of the present disclosure to an animal or human in need thereof. The composition or vaccine can be administered once as a single dose immunogenic composition or vaccine, or several times. When administered more than once, the second or subsequent doses will be administered at least 2, 3, 4, 5, 6, 7, 8, 9, 10, 11, 12, 13, 14, 15, 16, 17, 18, 19, 20, 21, 22, 23, 24, 25, 26, 27, 28, 29, 30 days, or more after the initial or previous administration. In preferred forms, the immune response will lessen the severity, frequency, and/or duration of at least one clinical sign of the disease by at least 10%, 20%, 30%, 40%, 50%, 60%, 70%, 80%, 90%, 95%, 96%, 97%, 98%, 99%, or even 100% in comparison to a group of animals or humans that did not receive an administration of the vaccine or immunogenic composition. Protection can include the complete prevention of clinical signs of infection, or a lessening of the severity, duration, or likelihood of the manifestation of one or more clinical signs of infection. Dosages may range, for example, from about 1 microgram to about 10,000 micrograms of the BVDV peptide per kg of the body weight of the subject receiving the administration. Methods are known in the art for determining or titrating suitable dosages of active antigenic agent to find minimal effective dosages based on the weight of the subject, concentration of the antigen and other typical factors.

In some preferred forms, said method also includes the administration of an immune stimulant. Preferably, said immune stimulant shall be given at least twice. Preferably, at least 3, more preferably at least 5, and even more preferably at least 7 days are between the first and the second or any further administration of the immune stimulant. Preferably, the immune stimulant is given at least 10 days, preferably 15, even more preferably 20, and still even more preferably at least 22 days beyond the initial administration of the immunogenic composition. It is understood that any immune stimulant known to a person skilled in the art can also be used. “Immune stimulant” as used herein, means any agent or composition that can trigger a general immune response, preferably without initiating or increasing a specific immune response, for example the immune response against a specific pathogen. It is further instructed to administer the immune stimulant in a suitable dose.

Desirably, the immunogenic composition or vaccine is administered to a subject not yet exposed to BVDV. The immunogenic composition or vaccine of the disclosure can conveniently be administered intranasally, transdermally (i.e., applied on or at the skin surface for systemic absorption), parenterally, etc. The parenteral route of administration includes, but is not limited to, intramuscular, intravenous, intraperitoneal, intradermal (i.e., injected or otherwise placed under the skin) routes and the like.

When administered as a liquid, the present immunogenic composition or vaccine may be prepared in the form of an aqueous solution, syrup, an elixir, a tincture and the like. Such formulations are known in the art and are typically prepared by dissolution of the antigen and other typical additives in the appropriate carrier or solvent systems. Suitable carriers or solvents include, but are not limited to, water, saline, ethanol, ethylene glycol, glycerol, etc. Typical additives are, for example, certified dyes, flavors, sweeteners and antimicrobial preservatives such as thimerosal (sodium ethylmercurithiosalicylate). Such solutions may be stabilized, for example, by addition of partially hydrolyzed gelatin, sorbitol or cell culture medium, and may be buffered by conventional methods using reagents known in the art, such as sodium hydrogen phosphate, sodium dihydrogen phosphate, potassium hydrogen phosphate, potassium dihydrogen phosphate, a mixture thereof, and the like.

Liquid formulations also may include suspensions and emulsions that contain suspending or emulsifying agents in combination with other standard co-formulants. These types of liquid formulations may be prepared by conventional methods. Suspensions, for example, may be prepared using a colloid mill. Emulsions, for example, may be prepared using a homogenizer.

Parenteral formulations, designed for injection into body fluid systems, require proper isotonicity and pH buffering to the corresponding levels of body fluids. Isotonicity can be appropriately adjusted with sodium chloride and other salts as needed. Suitable solvents, such as ethanol or propylene glycol, can be used to increase the solubility of the ingredients in the formulation and the stability of the liquid preparation. Further additives that can be employed in the present vaccine include, but are not limited to, dextrose, conventional antioxidants and conventional chelating agents such as ethylenediamine tetraacetic acid (EDTA). Parenteral dosage forms must also be sterilized prior to use.

A method for eliciting an immune response against BRD and/or clinical signs or symptoms of infection with BVDV is also provided. Such a method follows the same methodology as set forth above.

An “immunogenic or immunological composition” refers to a composition of matter that comprises at least one antigen which elicits an immunological response in the host of a cellular and/or antibody-mediated immune response to the composition or vaccine of interest. Usually, an “immunological response” includes but is not limited to one or more of the following effects: the production or activation of antibodies, B cells, helper T cells, suppressor T cells, and/or cytotoxic T cells and/or yd T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction in the severity or prevalence of, up to and including a lack of symptoms normally displayed by an infected host, a quicker recovery time and/or a lowered viral titer in the infected host.

The terms “immunogenic protein”, “immunogenic polypeptide” or “immunogenic amino acid sequence” as used herein refer to any amino acid sequence which elicits an immune response in a host against a pathogen comprising said immunogenic protein, immunogenic polypeptide or immunogenic amino acid sequence. An “immunogenic protein”, “immunogenic polypeptide” or “immunogenic amino acid sequence” as used herein, includes the full-length sequence of any proteins, analogs thereof, or immunogenic fragments thereof. By “immunogenic fragment” is meant a fragment of a protein which includes one or more epitopes and thus elicits the immunological response against the relevant pathogen. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, New Jersey. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996), J. Immunol. 157:3242-3249; Suhrbier, A. (1997), Immunol. and Cell Biol. 75:402-408; Gardner et al., (1998) 12th World AIDS Conference, Geneva, Switzerland, Jun. 28-Jul. 3, 1998. It is understood that immunogenic proteins of the present disclosure include the epitopes of SEQ ID NOS. 1-200.

In preferred forms, the BVDV peptide has 100% sequence identity and 100% sequence homology with the sequences disclosed herein. However, it is understood that some variation is possible without effecting the usefulness of the peptides in the immunogenic composition. Accordingly, the present disclosure also covers peptides having at least 85, 86, 87, 88, 89, 90, 91, 92, 93, 94, 95, 96, 97, 98, and 99% sequence homology and/or sequence identity with the peptides disclosed herein.

“Sequence Identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48:1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCVI NLM NIH Bethesda, MD 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 85%, preferably 90%, even more preferably 95% “sequence identity” to a reference nucleotide sequence, it is intended that the nucleotide sequence of the given polynucleotide is identical to the reference sequence except that the given polynucleotide sequence may include up to 15, preferably up to 10, even more preferably up to 5 point mutations per each 100 nucleotides of the reference nucleotide sequence. In other words, in a polynucleotide having a nucleotide sequence having at least 85%, preferably 90%, even more preferably 95% identity relative to the reference nucleotide sequence, up to 15%, preferably 10%, even more preferably 5% of the nucleotides in the reference sequence may be deleted or substituted with another nucleotide, or a number of nucleotides up to 15%, preferably 10%, even more preferably 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 85%, preferably 90%, even more preferably 95% sequence identity to a reference amino acid sequence, it is intended that the given amino acid sequence of the polypeptide is identical to the reference sequence except that the given polypeptide sequence may include up to 15, preferably up to 10, even more preferably up to 5 amino acid alterations per each 100 amino acids of the reference amino acid sequence. In other words, to obtain a given polypeptide sequence having at least 85%, preferably 90%, even more preferably 95% sequence identity with a reference amino acid sequence, up to 15%, preferably up to 10%, even more preferably up to 5% of the amino acid residues in the reference sequence may be deleted or substituted with another amino acid, or a number of amino acids up to 15%, preferably up to 10%, even more preferably up to 5% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

“Sequence homology”, as used herein, refers to a method of determining the relatedness of two sequences. To determine sequence homology, two or more sequences are optimally aligned, and gaps are introduced if necessary. However, in contrast to “sequence identity”, conservative amino acid substitutions are counted as a match when determining sequence homology. In other words, to obtain a polypeptide or polynucleotide having 95% sequence homology with a reference sequence, 85%, preferably 90%, even more preferably 95% of the amino acid residues or nucleotides in the reference sequence must match or comprise a conservative substitution with another amino acid or nucleotide, or a number of amino acids or nucleotides up to 15%, preferably up to 10%, even more preferably up to 5% of the total amino acid residues or nucleotides, not including conservative substitutions, in the reference sequence may be inserted into the reference sequence. Preferably the homologous sequence comprises at least a stretch of 50, even more preferably 100, even more preferably 250, even more preferably 500 nucleotides.

A “conservative substitution” refers to the substitution of an amino acid residue or nucleotide with another amino acid residue or nucleotide having similar characteristics or properties including size, hydrophobicity, etc., such that the overall functionality does not change significantly. In the case of a substitution, one or more consecutive or nonconsecutive amino acids are replaced by “equivalent” amino acids. The expression “equivalent” amino acid is directed here at designating any amino acid capable of being substituted by one of the amino acids of the base structure without, however, essentially modifying the biological activities of the corresponding peptides and such that they will be defined by the following. These equivalent amino acids can be determined either by depending on their structural homology with the amino acids which they substitute, or on results of comparative tests of biological activity between the different polypeptides, which are capable of being carried out. By way of example, the possibilities of substitutions capable of being carried out without resulting in an extensive modification of the biological activity of the corresponding modified polypeptides will be mentioned, the replacement, for example, of leucine by valine or isoleucine, of aspartic acid by glutamic acid, of glutamine by asparagine, of arginine by lysine etc., the reverse substitutions naturally being envisageable under the same conditions.

Isolated” means altered “by the hand of man” from its natural state, i.e., if it occurs in nature, it has been changed or removed from its original environment, or both. For example, a polynucleotide or polypeptide naturally present in a living organism is not “isolated,” but the same polynucleotide or polypeptide separated from the coexisting materials of its natural state is “isolated”, as the term is employed herein.

Another aspect of the present disclosure relates to a kit. Generally the kit includes a container comprising at least one dose of the immunogenic composition of BVDV peptide as provided herewith, wherein one dose comprises at least 2 μg of such peptide. Said container can comprise from 1 to 250 doses of the immunogenic composition. In some preferred forms, the container contains 1, 10, 25, 50, 100, 150, 200, or 250 doses of the immunogenic composition of the disclosure. Preferably, each of the containers comprising more than one dose of the immunogenic composition further comprises an anti-microbiological active agent, as described above. Those agents are for example, antibiotics including Gentamicin and Merthiolate and the like. Thus, one aspect of the present disclosure relates to a container that comprises from 1 to 250 doses of the immunogenic composition, wherein one dose comprises at least 2 μg BVDV peptide, and Gentamicin and/or Merthiolate, preferably from about 1 μg/ml to about 60 μg/ml of antibiotics, and more preferably less than about 30 μg/ml. In preferred forms, the kit also includes an instruction manual, including the information for the administration of at least one dose of the immunogenic composition into a susceptible animal, preferably selected from the group consisting of mammals, and still more preferably cattle, to treat, prevent, or lessen the incidence and/or severity of clinical symptoms associated with BVDV infection. Moreover, according to a further aspect, said instruction manual comprises the information of a second or further administration(s) of at least one dose of the immunogenic composition, wherein the second administration or any further administration is at least 14 days beyond the initial or any former administration. In some preferred forms, said instruction manual also includes the information, to administer an immune stimulant. Preferably, said immune stimulant shall be given at least twice. Preferably, at least 3, more preferably at least 5, and even more preferably at least 7 days are between the first and the second or any further administration of the immune stimulant. Preferably, the immune stimulant is given at least 10 days, preferably 15, even more preferably 20, and still even more preferably at least 22 days beyond the initial administration of the immunogenic composition. It is understood that any immune stimulant known to a person skilled in the art can also be used. “Immune stimulant” as used herein, means any agent or composition that can trigger a general immune response, preferably without initiating or increasing a specific immune response, for example the immune response against a specific pathogen. It is further instructed to administer the immune stimulant in a suitable dose. The kit may also comprise a second container, including at least one dose of the immune stimulant.

A further aspect relates to the use of any of the compositions provided herewith as a medicament, even more preferably as a vaccine. Moreover, the present disclosure also relates to the use of any of the compositions described herein, for the preparation of a medicament for lessening the severity of clinical symptoms associated with BVDV infection. Preferably, the medicament is for the prevention of a BVDV infection in mammals, preferably cattle.

A further aspect relates to a method for (1) the prevention of an infection, or re-infection with BVDV or (2) the reduction in incidence or severity of or elimination of clinical symptoms caused by BVDV in a subject, comprising administering any of the immunogenic compositions provided herewith to a subject in need thereof. Preferably, the subject is a mammal, and more preferably is cattle. It is understood that the reduction is in comparison to a subject that has not received an administration of a composition of the present disclosure. Preferably, one dose or at least two doses of the immunogenic composition is/are administered, wherein one dose preferably comprises at least about 2 μg BVDV peptide. A further aspect relates to the method of treatment as described above, wherein a subsequent application of the immunogenic composition is administered. Preferably, the second administration is done with the same immunogenic composition, preferably having the same amount of BVDV peptide. Preferably, the second administration is done at least 14 days beyond the initial administration, even more preferably at least 4 weeks beyond the initial administration. In preferred forms, the method is effective after just a single dose of the immunogenic composition and does not require a second or subsequent administration(s) in order to confer the protective benefits upon the subject.

It is understood that “prevention” as used in the present disclosure, includes the complete prevention of infection by a BVDV, but also encompasses a reduction in the severity of or incidence of clinical signs associated with or caused by BVDV. Such prevention is also referred to herein as a protective effect.

It should be appreciated that when typical reaction conditions (e.g., temperature, reaction times, etc.) have been given, the conditions both above and below the specified ranges can also be used, though generally less conveniently. The examples are conducted at room temperature (about 23° C. to about 28° C.) and at atmospheric pressure. All parts and percents referred to herein are on a weight basis and all temperatures are expressed in degrees centigrade unless otherwise specified. Further unless noted otherwise, all components of the disclosure are understood to be disclosed to cover “comprising”, “consisting essentially of”, and “consisting of” claim language as those terms are commonly used in patent claims.

The composition according to the disclosure may be applied intradermally, intratracheally, or intravaginally. The composition preferably may be applied intramuscularly or intranasally. In an animal body, it can prove advantageous to apply the pharmaceutical compositions as described above via an intravenous injection or by direct injection into target tissues. For systemic application, the intravenous, intravascular, intramuscular, intranasal, intraarterial, intraperitoneal, oral, or intrathecal routes are preferred. A more local application can be effected subcutaneously, intradermally, intracutaneously, intracardially, intralobally, intramedullarly, intrapulmonarily or directly in or near the tissue to be treated (connective-, bone-, muscle-, nerve-, epithelial tissue). Depending on the desired duration and effectiveness of the treatment, the compositions according to the disclosure may be administered once or several times, also intermittently, for instance on a daily basis for several days, weeks or months, and in different dosages.

### BRIEF DESCRIPTION OF THE DRAWINGS

FIG. 1 is a set of two graphs illustrating BVDV cross-reactive CD8⁺ T cell responses in immunized steers wherein in panel A) the purified CD8+ T cells and autologous CD14+ monocytes were incubated with gamma-irradiated BVDV-1b TGAC and in panel B) they were incubated with gamma-irradiated BVDV-2a;

FIG. 2 is a graph illustrating IFN-γ⁺CD8⁺ T cell responses by the predicted BVDV-1b peptide pools. CD8⁺ T cells from two steers that were immunized with BVDV-1b TGAC (2539 and 2599) and one steer immunized with BVDV-2a A125 (2593) (Table 2) were used to screen pools of predicted bovine MHC I-binding BVDV-1b peptides (Table 1) by IFN-γ ELISPOT. CD8⁺ T cells and autologous CD14⁺ monocytes were incubated with peptide pools [Pools 1 to 20] where, each pool contained 10 predicted peptides (Table 1). A pool of previously defined BVDV CD4⁺ T cell epitopes was included as a negative control. Responses are presented as spot forming cells (SFC) per million CD8⁺ T cells after the background media counts were deducted;

FIG. 3 is a set of 8 graphs illustrating IFN-γ-inducing CD8⁺ T cell epitopes from structural BVDV antigens. The predicted bovine MHC I-binding epitopes from BVDV-1b E^{rns}, E1, and E2 stimulated IFN-γ responses in CD8⁺ T cells from BVDV-immunized steers [TGAC-immunized: 2539 (●), 2565 (▴), 2599 (▪), and 2609 (♦); A125-immunized: 2593 (○), 2556 (Δ), 2601 (□), and 2611 (⋄)]. Responses are presented as spot forming cells (SFC) per million CD8⁺ T cells minus media background counts and bars represent the mean responses for the two groups;

FIG. 4 is a set of 8 graphs illustrating CD8⁺ T cell epitopes from BVDV non-structural N^{pro}, NS2, NS3, and NS4A antigens. IFN-γ⁺CD8⁺ T cell responses were stimulated in BVDV-immunized steers [TGAC-immunized: 2539 (●), 2565 (▴), 2599 (▪), and 2609 (♦); A125-immunized: 2593 (○), 2556 (Δ), 2601 (□), and 2611 (⋄)] by epitopes predicted from BVDV-1b N^{pro}, NS2, NS3, and NS4A non-structural antigens. Responses are presented as spot forming cells (SFC) per million CD8⁺ T cells minus media background counts and bars represent the mean responses for the two groups;

FIG. 5 is a series of 12 graphs illustrating BVDV NS4B-, NS5A- and NS5B-derived broadly reactive CD8⁺ T cell epitopes. CD8⁺ T cells from BVDV-immunized steers [TGAC-immunized: 2539 (●), 2565 (▴), 2599 (▪), and 2609 (♦); A125-immunized: 2593 (○), 2556 (Δ), 2601 (□), and 2611 (⋄)] recognized various highly conserved bovine MHC I-binding epitopes predicted from BVDV-1b NS4B, NS5A, and NS5B. Responses are presented as spot forming cells (SFC) per million CD8⁺ T cells minus media background counts and bars represent the mean responses for the two groups;

FIG. 6 is a set of two graphs illustrating that predicted CD8⁺ T cell epitopes from BVDV are bovine MHC I-restricted. Anti-bovine MHC I mAbs reduced IFN-γ⁺CD8⁺ T cell responses in two BVDV-immunized steers (2539 and 2593) against IFN-γ-inducing epitopes, N^{pro}₉₅₋₁₀₃, E^{rns}₄₉₃₋₅₀₁, E1₆₁₀₋₆₁₈, E2₉₉₉₋₁₀₀₇, NS4B₂₅₈₅₋₂₅₉₃, and NS5A₂₇₈₃₋₂₇₉₁ [Peptides 61, 86, 56, 100, 37, and 64 respectively (Table 3)]. CD8⁺ T cells and autologous CD14⁺ monocytes were incubated with the individual peptides either in the presence or absence of anti-bovine MHC I mAbs. Responses are presented as spot forming cells (SFC) per million CD8⁺ T cells minus media background counts and bars represent the mean responses for the two steers. Statistically significant differences between the responses of steers due to MHC I blockade is indicated by asterisks (*p<0.05);

FIG. 7 is an illustration of the mutant BPI3V TVMDL16 sequence;

FIG. 8 is an illustration of the BPIV3Vc-E2 backbone;

FIG. 9 is an illustration of the optimized T7 polymerase gene in pCAGGSS;

FIG. 10 is an illustration of an attenuated BPI3Vc-E2^{b} virus expressing the E2^{b} transgene;

FIG. 11A is a photograph illustrating the surface display of a BVDV E2^{b} transgene on cells infected with BPI3Vc-E2^{b} virus;

FIG. 11B is a photograph illustrating the surface display of a BVDV E2^{b} transgene on cells infected with BPI3Vc-E2^{b} virus;

FIG. 11C is a photograph illustrating the surface display of a BVDV E2^{b} transgene on cells infected with BPI3Vc-E2^{b} virus;

FIG. 11D is a photograph illustrating the surface display of a BVDV E2^{b} transgene on cells infected with BPI3Vc-E2^{b} virus;

FIG. 12A is a photograph illustrating the authenticity of mosaic BPI3V F2-HN2 expressed by plasmid constructs;

FIG. 12B is a photograph illustrating the authenticity of mosaic BPI3V F2-HN2 expressed by plasmid constructs;

FIG. 12C is a photograph illustrating the authenticity of mosaic BPI3V F2-HN2 expressed by plasmid constructs; and

FIG. 12D is a photograph illustrating the authenticity of mosaic BPI3V F2-HN2 expressed by plasmid constructs.

### DETAILED DESCRIPTION OF THE DISCLOSURE

This written description uses examples to disclose the subject matter of the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

### EXAMPLE 1

### Materials and Methods

### BVDV 9-Mer Peptide Prediction and Synthesis

A BVDV-1b strain was chosen for BVDV CD8+ T cell epitope mapping since it's the predominant sub-genotype in the United States. For epitope prediction, the BVDV-1b polyprotein sequence (GenBank: AGG54029.1) was used as the input sequence and 9-mer peptide length along with all the available BoLA I alleles in the NetMHCpan2.8 database, which can be found on the internet at cbs.dtu.dk/services/NetMHCpan-2.8, were selected. The predicted 9-mers were then sorted by their prediction scores. Overall, two-hundred candidate epitopes were selected that were predicted as strong binders for their corresponding predicted BoLA I alleles (Table 1). The two-hundred peptide sequences were used to generate a library of crude synthetic 9-mer peptides (Peptide 2.0, Inc.). Each synthetic peptide was re-constituted at a concentration of 10 mg/ml in ultrapure sterile water with 25% DMSO.

TABLE I
Bovine MHC I-binding 9-mer peptides from BVDV-1b polyprotein
predicted using NetMHCpan version 2.8

| Pool 1 | | Pool 2 | | Pool 3 | | Pool 4 | | Pool 5 | |
|---|---|---|---|---|---|---|---|---|---|
| Peptide | | Peptide | | Peptide | | Peptide | | Peptide | |
| SEQ ID | | SEQ ID | | SEQ ID | | SEQ ID | | SEQ ID | |
| NO | Sequence | NO | Sequence | NO | Sequence | NO | Sequence | NO | Sequence |
| 1 | FQRGVNRSL | 11 | YLPYATSAL | 21 | YQDYKGPVY | 31 | AQFGAGEIV | 41 | RQAAVDLVV |
| 2 | VQYTARGQL | 12 | YMAGRDTAV | 22 | LMISYVTDY | 32 | SEVLLLSVV | 42 | LQLQTRTSL |
| 3 | LTIPNWRPL | 13 | KQMSLTPLF | 23 | AENALLIAL | 33 | LEQEVQVEI | 43 | IMFEAFELL |
| 4 | VASLFISAL | 14 | AMVEYSYIF | 24 | YEMKALRNV | 34 | YETATVLVF | 44 | KAVAFSFLL |
| 5 | YILDLIYSL | 15 | FAPETASVL | 25 | YEYSDGLQL | 35 | QEYSGFVQY | 45 | FEEASMCEI |
| 6 | LLMYSWNPL | 16 | QQYMLKGEY | 26 | WQMVYMAYL | 36 | RQLGILGKK | 46 | FEIAVSDVL |
| 7 | GEYQYWFDL | 17 | YQYWFDLEI | 27 | SQFLDIAGL | 37 | SEQKRTLLM | 47 | YAASPYCEV |
| 8 | REMNYDWSL | 18 | YMAYLTLDF | 28 | RTYKRVRPF | 38 | REHNKWILK | 48 | TAATTTAFL |
| 9 | TAFFGVMPR | 19 | YMLKGEYQY | 29 | YKRWIQCVL | 39 | AMAVLTLTL | 49 | WPYETATVL |
| 10 | SALATYTYK | 20 | NMMDKLTAF | 30 | RDYFAESLL | 40 | ALRDFNPEL | 50 | IPNWRPLTF |
| Pool 6 | | Pool 7 | | Pool 8 | | Pool 9 | | Pool 10 | |
| Peptide | | Peptide | | Peptide | | Peptide | | Peptide | |
| SEQ ID | | SEQ ID | | SEQ ID | | SEQ ID | | SEQ ID | |
| NO | Sequence | NO | Sequence | NO | Sequence | NO | Sequence | NO | Sequence |
| 51 | LAALHTRAL | 61 | GPVYHRAPL | 71 | YHIIVMHPL | 81 | KTARNINLY | 91 | MLNVLTMMY |
| 52 | NPLVRRICL | 62 | YAIAKNDEI | 72 | QLFLRNLPI | 82 | ISSKWQMVY | 92 | YTARGQLFL |
| 53 | ITYASYGYF | 63 | SVMLGVGAI | 73 | KLANLNLSL | 83 | ISSKTGHLY | 93 | SSAENALLI |
| 54 | YTMKLSSWF | 64 | YYDDNLNEI | 74 | APVRFPTAL | 84 | KSWLGGLDY | 94 | YLKPGPLFY |
| 55 | SVIQDTAHY | 65 | FVNEDIGTI | 75 | YIPDKGYTL | 85 | ITLATGAGK | 95 | KVVEPALAY |
| 56 | GSVWNLGKY | 66 | ARRVKIHPY | 76 | VILSTTIYK | 86 | FGAYAASPY | 96 | ETASVLYLV |
| 57 | SVYQYMRLK | 67 | LRRLRVLLM | 77 | ATVTTWLAY | 87 | KGYNSGYYY | 97 | WADFLTLIL |
| 58 | STQTTYYYK | 68 | DTYENYSFL | 78 | ISALATYTY | 88 | KSKTWFGAY | 98 | RVIAALIEL |
| 59 | WTAATTTAF | 69 | VMSRVIAAL | 79 | VAFSFLLMY | 89 | RYYETAIPK | 99 | ALFEAVOTI |
| 60 | NSMLNVLTM | 70 | GHMASAYQL | 80 | KVLKWVHNK | 90 | SRDERPFVL | 100 | YFEPRDNYF |
| Pool 11 | | Pool 12 | | Pool 13 | | Pool 14 | | Pool 15 | |
| Peptide | | Peptide | | Peptide | | Peptide | | Peptide | |
| SEQ ID | | SEQ ID | | SEQ ID | | SEQ ID | | SEQ ID | |
| NO | Sequence | NO | Sequence | NO | Sequence | NO | Sequence | NO | Sequence |
| 101 | YGMPKVVTI | 111 | FGPGVDAAM | 121 | TTATVRELL | 131 | AGNSMLNVL | 141 | LTLDFMYYM |
| 102 | VVTYFLLLY | 112 | WRPLTFILL | 122 | ENALLIALF | 132 | IGPLGATGL | 142 | EGRRFVASL |
| 103 | YSYIFLDEY | 113 | YSFLNARKL | 123 | VTTWLAYTF | 133 | CTFNYTRTL | 143 | RGLETGWAY |
| 104 | KIMGAISDY | 114 | LLPLIRATL | 124 | TPSDERIRL | 134 | DSIEVVTYF | 144 | IGNPLRLIY |
| 105 | IAYEKAVAF | 115 | ATPEQLAVI | 125 | HPYEAYLKL | 135 | DSKLYHIYV | 145 | TTTAFLVCL |
| 106 | VTGSDSKLY | 116 | VTIIRACTL | 126 | RGKFNTTLL | 136 | RGDFKQITL | 146 | VSVGISVML |
| 107 | VTASGTPAF | 117 | FGYVGYQAL | 127 | KGWSGLPIF | 137 | LGPIVNLLL | 147 | TTLLNGPAF |
| 108 | ATTVVRTYK | 118 | YNIEPWILL | 128 | HGWCNWYNI | 138 | MTATPAGSV | 148 | DTKSFHEAI |
| 109 | TSMNRGDFK | 119 | DNYFQQYML | 129 | AGVFLIRSL | 139 | NSYEVQVPV | 149 | SLTPLFEEL |
| 110 | KGPVSGIYL | 120 | MVYMAYLTL | 130 | TYFLLLYLL | 140 | ESGEGVYLF | 150 | KIHPYEAYL |
| Pool 16 | | Pool 17 | | Pool 18 | | Pool 19 | | Pool 20 | |
| Peptide | | Peptide | | Peptide | | Peptide | | Peptide | |
| SEQ ID | | SEQ ID | | SEQ ID | | SEQ ID | | SEQ ID | |
| NO | Sequence | NO | Sequence | NO | Sequence | NO | Sequence | NO | Sequence |
| 151 | KNFSFAGIL | 161 | KLLEIFHTI | 171 | ALRNVSGSL | 181 | REALEALSL | 191 | VNYRVTKYY |
| 152 | KSFNRVARI | 162 | GTAKLTTWL | 172 | MEILSQNPV | 182 | LTPLFEELL | 192 | AMFQRGVNR |
| 153 | YHRAPLELF | 163 | LAQGNWEPL | 173 | IEFCSHTPV | 183 | GEIVMMGNL | 193 | LSSAENALL |
| 154 | LLAWAILAL | 164 | YLERVDLSF | 174 | KEHDCTSVI | 184 | SEKHLVEQL | 194 | ALRYVAGPI |
| 155 | KLMSGIQTV | 165 | WSDNTSSYM | 175 | AESLLVIVV | 185 | YELVKLYYL | 195 | GIYLKPGPL |
| 156 | RRFVASLFI | 166 | VIPGSVWNL | 176 | LMNKTQANL | 186 | SQNPVSVGI | 196 | GENITQWNL |
| 157 | KMLLATDKW | 167 | MMDKLTAFF | 177 | ALSKRHVPM | 187 | ITGAQGFPY | 197 | RECAVTCRY |
| 158 | IYLKPGPLF | 168 | YMRLKHPSI | 178 | AMDDKLGPM | 188 | ALIELNWTM | 198 | GRHKRVLVL |
| 159 | YEKAVAFSF | 169 | LLRRLRVLL | 179 | GLWGTHTAL | 189 | RETRYLAAL | 199 | ILLQGAPVL |
| 160 | ALLGGRYVL | 170 | VOKFINSLI | 180 | GEDLYDCAL | 190 | GVFLIRSLK | 200 | ASYGYFCQM |

### Inactivation of BVDV by Gamma-Irradiation

BVDV-1b TGAC and BVDV-2a A125 were inactivated by gamma-irradiation at The Kansas State University TRIGA Mark II nuclear reactor facility, as described previously. Briefly, 1 ml (1.5×1010 TCID50) of each virus was irradiated with an estimated dose of 200 krad using Californium-252 source. To ensure inactivation of BVDV, the viability of the gamma-irradiated viruses was tested by infecting MDBK cells and the presence of virus progenies was evaluated using BVDV-specific antibodies. Briefly, following 72 hours of incubation at 37° C., the cells were observed for CPE and the culture supernatant were collected. Fresh MDBK cells were then exposed to the collected supernatant and were incubated for another 72 hours. For detection of rescued viral particles, after fixing, the cells were stained with anti-BVDV polyclonal sera (Porcine origin, Cat #210-70-BVD, VMRD, Inc) and alkaline phosphatase conjugated goat anti-porcine IgG (Jackson ImmunoResearch, Cat #114-055-003) whereby no BVDV-positive cells were detected (data not shown).

### Infection and Immunization of Steers

Eight, seven-eight months old, BVDV-1 and -2 seronegative steers were infected intranasally with BVDV-1b CA0401186a strain (52). After four weeks, following recovery, the steers were randomly allocated into two groups A-B (n=4) (Table 2). Steers in both groups were boosted six times every four weeks with gamma-irradiated BVDV-1b TGAC or BVDV-2a A125 (Table 2). Gamma-irradiated BVDV mixed with MONTANIDETM ISA 201 VG adjuvant (Seppic) was administered intramuscularly in the neck region. During immunization, weekly sera and peripheral blood mononuclear cells (PBMCs) samples were collected. At four weeks after the last boost, the steers were bled, and spleens were collected after the animals were euthanized.

TABLE 2
Immunization of steers that were previously
exposed to BVDV-1b CA401186a.

| | Steer ID and | | Immunization | No. of |
|---|---|---|---|---|
| Groups | Figure Legend | Immunogen | Dose/Steer | Immunizations |
| A: | 2539 ● | Gamma | 200 μg | 6 |
| TGAC | 2565 ▴ | irradiated | | |
| | 2599 ▪ | BVDV-1b | | |
| | 2609 ♦ | TGAC | | |
| B: | 2593 ∘ | Gamma | 200 μg | 6 |
| A125 | 2556 Δ | irradiated | | |
| | 2601 □ | BVDV-2a | | |
| | 2611 ⋄ | A125 | | |

### CD8+ T Cell and Autologous CD14+ Monocyte Isolation

For all BVDV-immunized steers, positively selected CD8+ T cells and autologous CD14+ monocytes were purified using MACS LS columns (Miltenyi Biotec, Cat #130-042-401) in accordance with vendor's protocol and as previously described. Anti-bovine CD8a mAb [7C2B clone, IgG2a isotype; WSU Monoclonal Antibody Center (WSUMAC), Item #BOV2019] and goat anti-mouse IgG microbeads (Miltenyi Biotec, Cat #130-048-402) were used for isolation of CD8+ T cells from splenocytes. Similarly, anti-bovine CD14 mAb (MM61A clone, IgG1 isotype; WSUMAC, Item #BOV2109), along with goat anti-mouse IgG microbeads, was used for the isolation of CD14+ monocytes from autologous PBMCs. The purity of the isolated subsets were determined to be 95-98% by flow cytometry (data not shown). Purified cell subsets were re-suspended in complete RPMI 1640 medium at appropriate dilution for IFN-γ ELISPOT assay.

### Evaluation of BVDV-Specific CD8+ T Cell Responses

IFN-γ responses in purified CD8+ T cells from the BVDV-immunized steers were evaluated by ELISPOT assay (Bovine IFN-γ ELISpot BASIC ALP kit, Mabtech, Cat #3119-2A) as in accordance with vendor's protocol and as previously described. Briefly, for all eight steers, 0.2×106 CD8+ T cells were co-cultured with 0.4×105 autologous CD14+ monocytes that were pulsed with 2.5 μg/ml of gamma-irradiated BVDV-1b TGAC or BVDV-2a A125 in a total volume of 100 μl complete RPMI 1640 medium in triplicate wells of MultiScreen-IP plates (MilliporeSigma™, Cat #MAIPS4510). Similar co-cultures incubated with 2.5 μg/ml of ConA or the medium alone served as positive and negative controls, respectively. The plates were incubated at 37° C. for 48 h and following processing, IFN-γ spots were enumerated using ELISPOT reader [ImmunoSpot® S6 Analyzer, Cellular Technology Limited]. The responses were reported as spot forming cells (SFC) per million CD8+ T cells after the background spot counts from negative control triplicates were deducted.

### Ex Vivo Screening of Predicted Bovine MHC I-Binding BVDV Peptides

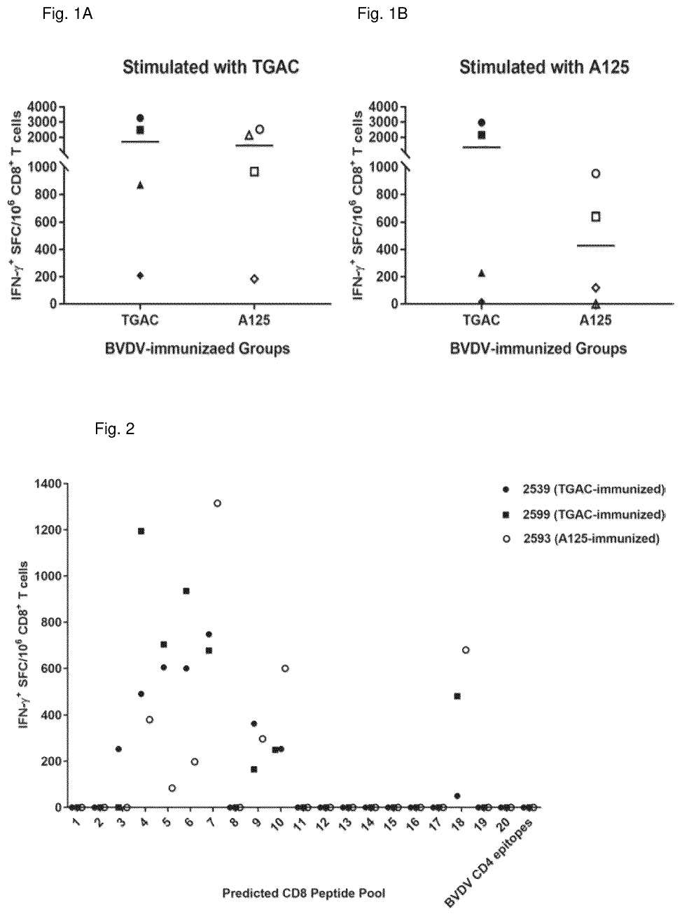

To screen the two-hundred predicted peptides, twenty pools of 10 peptides were generated and each peptide was diluted to a final concentration of 2.5 μg/ml in complete RPMI 1640 medium (Table 1). The peptide pools were tested for non-specific IFN-γ responses using PBMCs collected from naïve steers and no background responses were detected (data not shown). Two steers (2539 and 2599) immunized with TGAC and one steer (2593) immunized with A125 had the highest number of TGAC- and A125-specific IFN-γ+CD8+ T cells, respectively, [FIG. 1] and thus, CD8+ T cells and autologous CD14+ monocytes purified from these steers were used to screen the 20 peptide pools by ELISPOT assay as described above. Additionally, a pool of previously defined BVDV CD4+ T cell epitope peptides (2.5 μg/ml of each peptide) was used as negative control. Peptide pools 4-7, 9, 10, and 18, stimulated IFN-γ+CD8+ T cells in the three steers and thus, individual peptides were tested to identify T cell epitopes. Peptides that generated IFN-γ+CD8+ T cell responses in 6-8 BVDV-immunized steers were reported as IFN-γ-inducing CD8+ T cell epitopes (Table 3).

TABLE 3
IFN-γ-inducing CD8⁺ T cell epitopes predicted from BVDV-1b polyprotein.

| Peptide | | | Conserved in BVDV | Predicted | Predicted |
|---|---|---|---|---|---|
| SEQ | Peptide | | Genotypes (number | BOLA I | 1- |
| ID NO. | Name* | Sequence | of strains) | Allele | log50k(aff) |
| 61 | N^{pro}₉₅₋₁₀₃ | GPVYHRAPL | 1a (6), 1b (7), 2a (9) | BOLA- | 0.474 |
| | | | | 2*03001 | |
| 45 | N^{pro}₁₀₆₋₁₁₄ | FEEASMCEI | 1a (6), 1b (7), 2a (9) | BOLA- | 0.317 |
| | | | | 1*07401 | |
| 176 | ^{Erns}₃₆₃₋₃₇₁ | LMNKTQANL | 1a (6), 1b (7), 2a (9) | BOLA- | 0.305 |
| | | | | 6*01501 | |
| 88 | E^{rns}₄₈₈₋₄₉₆ | KSKTWFGAY | 1a (6), 1b (7), 2a (9) | BOLA- | 0.366 |
| | | | | 2*04601 | |
| 86 | E^{rns}₄₉₃₋₅₀₁ | FGAYAASPY | 1a (6), 1b (7), 2a (9) | BOLA- | 0.388 |
| | | | | 2*04601 | |
| 47 | E^{rns}₄₉₆₋₅₀₄ | YAASPYCEV | 1a (6), 1b (7), 2a (9) | BOLA- | 0.344 |
| | | | | 2*00501 | |
| 32 | E1₅₅₂₋₅₆₀ | SEVLLLSVV | 1a (6), 1b (7) | BOLA- | 0.523 |
| | | | | 1*01901 | |
| 56 | E1₆₁₀₋₆₁₈ | GSVWNLGKY | 1a (6), 1b (7), 2a (9) | BOLA- | 0.292 |
| | | | | 2*00801 | |
| 34 | E1₆₂₈₋₆₃₆ | YETATVLVF | 1b (7) | BOLA- | 0.292 |
| | | | | 1*07401 | |
| 100 | E2₉₉₉₋₁₀₀₇ | YFEPRDNYF | 1a (44), 1b (51), 2 | BOLA- | 0.141 |
| | | | (112) | 2*06001 | |
| 39 | NS2₁₁₉₅₋₁₂₀₃ | AMAVLTLTL | 1a (44), 1b (51) | BOLA- | 0.490 |
| | | | | 1*04901 | |
| 97 | NS2₁₂₉₁₋₁₂₉₉ | WADFLTLIL | 1a (44), 1b (51), | BOLA- | 0.213 |
| | | | 2 (112) | 2*05601 | |
| 82 | NS2₁₃₇₃₋₁₃₈₁ | ISSKWQMVY | 1a (44), 1b (51), | BOLA- | 0.434 |
| | | | 2 (112) | 2*04301 | |
| 69 | NS2₁₄₀₇₋₁₄₁₅ | VMSRVIAAL | 1a (44), 1b (51) | BOLA- | 0.343 |
| | | | | 2*02601 | |
| 87 | NS3₂₀₁₀₋₂₀₁₈ | KGYNSGYYY | 1a (44), 1b (51), | BOLA- | 0.408 |
| | | | 2 (112) | 2*04601 | |
| 177 | NS4A₂₂₉₁₋₂₂₉₉ | ALSKRHVPM | 1 (77), 2 (101) | BOLA- | 0.327 |
| | | | | 6*01501 | |
| 172 | NS4B₂₅₅₅₋₂₅₆₃ | MEILSQNPV | 1 (77), 2 (101) | BOLA- | 0.450 |
| | | | | 6*01401 | |
| 63 | NS4B₂₅₆₈₋₂₅₇₆ | SVMLGVGAI | 1 (77), 2 (101) | BOLA- | 0.257 |
| | | | | 2*01801 | |
| 37 | NS4B₂₅₈₅₋₂₅₉₃ | SEQKRTLLM | 1 (77), 2 (101) | BOLA- | 0.509 |
| | | | | 1*04201 | |
| 99 | NS4B₂₆₂₀₋₂₆₂₈ | ALFEAVQTI | 1 (77), 2 (101) | BOLA- | 0.246 |
| | | | | 2*05701 | |
| 43 | NS4B₂₆₆₄₋₂₆₇₂ | IMFEAFELL | 1 (77), 2 (101) | BOLA- | 0.362 |
| | | | | 1*06701 | |
| 64 | NS5A₂₇₈₃₋₂₇₉₁ | YYDDNLNEI | 1 (77), 2 (101) | BOLA- | 0.247 |
| | | | | 2*01802 | |
| 65 | NS5A₂₉₉₂₋₂₉₃₀ | FVNEDIGTI | 1 (77), 2 (101) | BOLA- | 0.218 |
| | | | | 2*01802 | |
| 81 | NS5A₃₀₃₈₋₃₀₄₆ | KTARNINLY | 1 (77), 2 (101) | BOLA- | 0.403 |
| | | | | 2*04501 | |
| 40 | NS5A₃₀₆₇₋₃₀₇₅ | ALRDFNPEL | 1 (77) | BOLA- | 0.448 |
| | | | | 1*04901 | |
| 38 | NS5B₃₂₇₃₋₃₂₈₁ | REHNKWILK | 1 (77), 2 (101) | BOLA- | 0.421 |
| | | | | 1*04201 | |
| 89 | NS5B₃₄₃₄₋₃₄₄₂ | RYYETAIPK | 1 (77), 2 (101) | BOLA- | 0.278 |
| | | | | 2*04701 | |
| 173 | NS5B₃₆₇₃₋₃₆₈₁ | IEFCSHTPV | 1 (77), 2 (101) | BOLA- | 0.454 |
| | | | | 6*01401 | |

*Peptide name represents the BVDV antigen and amino acid position for the predicted peptide within BVDV-1b polyprotein.

### IFN-γ-Inducing CD8+ T Cell Epitope Sequences Analyses

Amino acid sequences of IFN-γ-inducing CD8+ T cell epitopes were evaluated for conservation across the two BVDV genotypes using National Center for Biotechnology Information Basic Local Alignment Search Tool (NCBI BLAST) (Table 3). CD8+ T cell epitopes derived from Npro, Erns, and E1 antigens were analyzed across six BVDV-1a, seven BVDV-1b, and nine BVDV-2a strains using available genome data in NCBI server. Similarly, for CD8+ T cell epitopes derived from E2, NS2-3, NS4A-B, and NS5A-B antigens, sequences were analyzed using the latest available BVDV genomes and published amino acid sequences of the individual BVDV antigens from different isolates whose full genomes have not been sequenced. Sequences from forty-four [44] BVDV-1a, fifty-one [51] BVDV-1b, and one hundred and twelve [112] BVDV-2 (all available BVDV-2 sub-genotypes were included) strains were used for the analyses of E2- and NS2-3-derived epitopes (Table 3). In the case of epitopes from NS4A-B and NS5A-B antigens, sequences from seventy-seven [77] BVDV-1 and one hundred and one [101] BVDV-2 strains from diverse sub-genotypes were analyzed (Table 3).

### MHC I Blocking ELISPOT Assay

The identified CD8+ T cell epitopes were tested for bovine MHC I-restriction by ELISPOT assay as above, but peptide binding was blocked with anti-bovine MHC I mAbs, H58A (IgG2a isotype; WSUMAC, Item #BOV2001) and PT85A (IgG2a isotype; WSUMAC, Item #BOV2002), at 1.0 μg/ml concentration (56). Six IFN-γ inducing CD8+ T cell epitopes, Npro95-103, Erns493-501, E1610-618, E2999-1007, NS4B2585-2593, and NS5A2783-2791 [Peptides 61, 86, 56, 100, 37, and 64, respectively (Table 3)], were selected for this assay. Co-cultures of CD8+ T cells and autologous CD14+ monocytes from one TGAC-immunized steer (2539) and one A125-immunized steer (2593), were incubated with 2.5 μg/ml of peptide in the presence of either the two anti-bovine MHC I mAbs or IgG2a isotype control. The IFN-γ+CD8+ T cell responses in the presence or absence of anti-bovine MHC I mAbs were reported as SFC per million CD8+ T cells described as above.

### Statistical Analysis

The results from MHC I blocking ELISPOT assay were analyzed by Wilcoxon test in GraphPad Prism 7 (Version 7.04, GraphPad Software, Inc. La Jolla, CA). The significance of the difference in peptide-specific IFN-γ+CD8+ T cell responses in the absence or presence of anti-bovine MHC I mAbs was determined by a non-parametric test and the level of significance was set at p<0.05.

### Results

### Gamma-Irradiated BVDV Primed and Expanded Strong Cross-Reactive IFN-γ+CD8+ T Cells in Steers

Steers that had previously recovered from BVDV-1b CA401186a infection were hyper-immunized with gamma-irradiated BVDV-1b TGAC or BVDV-2a A125 (Table 2). All the steers had high levels of BVDV-specific IFN-γ-secreting CD8+ T cells in their splenocytes (FIG. 1). Strong TGAC-specific IFN-γ+CD8+ T cell responses were detected in the spleens of all the steers immunized with TGAC or A125 (FIG. 1, panel A), whereas A125-specific IFN-γ+CD8+ T cells were detected in 4/4 TGAC vaccinees and in 3/4 A125 vaccinees (FIG. 1, panel B). Multiple boosts of the steers with the gamma-irradiated BVDV-1 or -2 successfully expanded robust BVDV-specific CD8+ T cells. These cells were cross-reactive as judged by strong recall of IFN-γ responses against TGAC as well as A125 BVDV strains.

### Predicted Bovine MHC I-Binding BVDV Peptides Stimulated IFN-γ+CD8+ T Cells

Pools of predicted bovine MHC I-binding 9-mer peptides from BVDV-1b polyprotein (Table 1) were tested for their ability to stimulate IFN-γ secretion by CD8+ T cells from the BVDV immunized steers (FIG. 2). IFN-γ responses against various peptide pools were detected in purified CD8+ T cells from three steers (two TGAC-immunized steers and one A125-immunized steer) that had the highest number of TGAC-as well as A125-specific IFN-γ+CD8+ T cells (FIG. 2). Among peptide pools 1 to 20, high levels of IFN-γ-secreting CD8+ T cells were stimulated by peptide pools: 4-7, 9, 10, and 18 in all the three steers (FIG. 2). As expected, the pool of defined BVDV CD4+ T cell epitopes did not recall IFN-γ+CD8+ T cells in steers since these epitopes are MHC-DR-restricted (FIG. 2). Consequently, individual peptides from the peptide pools that stimulated IFN-γ+CD8+ T cell responses were further evaluated to identify IFN-γ-inducing BVDV CD8+ T cell epitopes.

### Structural BVDV Antigens Contain Novel IFN-γ-Inducing CD8+ T Cell Epitopes

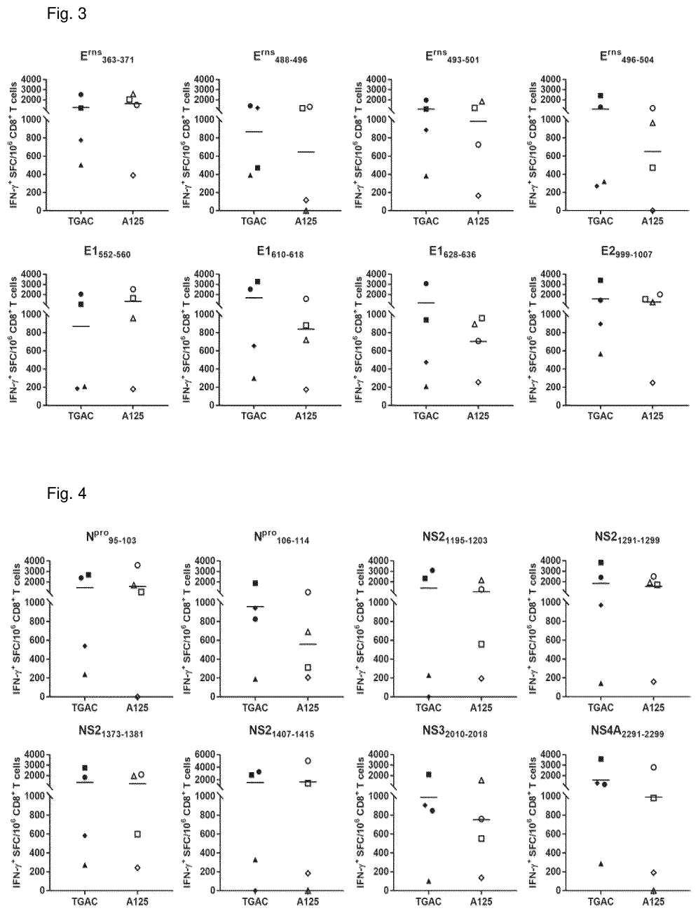

Eight bovine MHC I-binding epitopes were identified from BVDV-1b structural antigens: Erns, E1 and E2 (Table 3), which were recognized by CD8+ T cells isolated from the spleens of BVDV-1b and -2a immunized steers (FIG. 3). Erns363-371, Erns488-496, Erns493-501, and Erns496-504, stimulated IFN-γ+CD8+ T cell responses in most of the TGAC and A125 vaccinees (FIG. 3). The Erns488-496, Erns493-501, and Erns496-504 are overlapping epitopes and interestingly, Erns488-496 and Erns493-501 were predicted as binders for the same BOLA I allele (Table 3). However, unlike the Erns488-496 epitope, the Erns493-501 epitope stimulated IFN-γ+CD8+ T cell responses in 4/4 TGAC- and A125-immunized steers (FIG. 3). Overall, out of the four Erns-derived epitopes, the Erns363-371 stimulated the highest number of IFN-γ+CD8+ T cells in 4/4 steers from both groups (FIG. 3). All BVDV-immunized steers also had IFN-γ+CD8+ T cell responses against the three epitopes from E1, E1552-560, E1610-618, and E1628-636 (FIG. 3). Among the predicted epitopes from the most immunogenic BVDV E2 antigen, only one IFN-γ-inducing CD8+ T cell epitope, E2999-1007, was identified (FIG. 3). Notably, this epitope stimulated very high levels of IFN-γ+CD8+ T cell responses in 4/4 TGAC-as well as A125-immunized steers (FIG. 3).

Upon analysis, it was determined that the Erns-derived epitopes (Erns363-371, Erns488-496, Erns493-501, and Erns496-504) are present in BVDV-1a, -1b, and -2a strains (Table 3). Hence, cross-reactive BVDV-specific CD8+ T cells were recalled by the epitopes in steers (FIG. 3). Similarly, E1610-618 recalled cross-reactive CD8+ T cells in immunized steers because it is present in BVDV-1a, -1b, and -2a strains (FIG. 3) (Table 3). E1552-560 epitope is specific to BVDV-1a and -1b strains, whereas E1628-636 epitope is only present in BVDV-1b strains (Table 3). Surprisingly, these two epitopes from E1 recalled CD8+ T cell responses in BVDV-2a immunized steers (FIG. 3). Since these steers were infected with BVDV-1b CA401186a prior to immunization, the two epitopes apparently recalled the E1-specific CD8+ T cell memory responses primed during infection (FIG. 3). Importantly, E2999-1007 was highly conserved across the 207 strains from BVDV-1a, -1b, and -2 genotypes (Table 3) and therefore, can prime bovine CD8+ T cells against diverse BVDV isolates (FIG. 3).

### Nonstructural BVDV Antigens Contain Multiple Novel Broadly Reactive CD8+ T Cell Epitopes

Novel T cell epitopes from the nonstructural antigens: Npro, NS2, NS3, and NS4A stimulated recall IFN-γ+ T cell responses in CD8+ T cells from BVDV-immunized steers (FIG. 4). Npro95-103 epitope recalled higher mean IFN-γ+CD8+ T cells in TGAC and A125 vaccinees than Npro106-114, but Npro106-114 epitope-specific recall responses were detected in all vaccinees (FIG. 4). The two Npro-derived epitopes are cross-reactive since they are present in BVDV-1a, -1b, and -2a strains (Table 3). Four IFN-γ-inducing CD8+ T cell epitopes were identified from NS2 antigen among which, NS21195-1203 and NS21407-1415 are conserved across 95 BVDV-1a and -1b strains, while NS21291-1299 and NS21373-1381 are conserved across the 207 BVDV-1a, -1b, and -2 strains (Table 3). In the TGAC treatment group, 3/4 steers had NS21195-1203- and NS21407-1415-specific recall of IFN-γ+CD8+ T cell responses (FIG. 4). On the other hand, NS21195-1203- and NS21407-1415-specific IFN-γ+CD8+ T cells were recalled in 4/4 and 3/4 A125 vaccinees, respectively, evidently due to the memory responses induced during BVDV-1b infection (FIG. 4). IFN-γ+CD8+ T cells were recalled by the two cross-reactive epitopes, NS21291-1299 and NS21373-1381, in 4/4 steers from both groups (FIG. 4). Likewise, NS32010-2018, an NS3-derived CD8+ T cell epitope which is also highly conserved across the 207 BVDV strains (Table 3), recalled IFN-γ+CD8+ T cells in 4/4 TGAC and A125 vaccinees (FIG. 4). An NS4A-derived epitope, NS4A2291-2299, conserved among 178 BVDV-1 and -2 strains (Table 3), recalled IFN-γ+CD8+ T cells in 4/4 TGAC and 3/4 A125 vaccinees (FIG. 4).

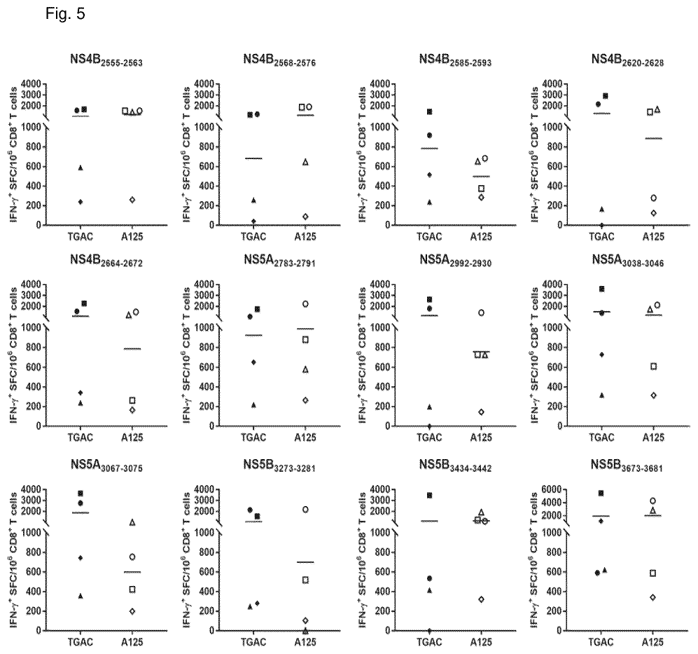

Various CD8+ T cell epitopes, which recalled IFN-γ+CD8+ T cell responses in BVDV-1b- and -2a-immunized steers, were located within NS4B, NS5A, and NS5B antigens (FIG. 5). With the exception of NS5A3067-3075, these epitopes were well-conserved across the 77 BVDV-1 and the 101 BVDV-2 strains (Table 3). The five NS4B-derived epitopes (NS4B2555-2563, NS4B2568-2576, NS4B2585-2593, NS4B2620-2628, and NS4B2664-2672) recalled IFN-γ+CD8+ T cells in all vaccinees except for one TGAC vaccinee, which had no detectable response against NS4B2620-2628 (FIG. 5). NS5A2783-2791, NS5A2992-2930, and NS5A3038-3046, like NS4-derived epitopes, induced IFN-γ recall responses in CD8+ T cells from the majority of BVDV-immunized steers (FIG. 5). The NS5A3067-3075epitope was present only in the BVDV-1 strains (Table 3) however, it recalled IFN-γ+CD8+ T cells in 4/4 TGAC and A125 vaccinees (FIG. 5). As observed for the other BVDV-1 specific CD8+ T cell epitopes, the NS5A3067-3075 epitope likely recalled BVDV-1b-specific memory responses in A125 vaccinees (FIG. 5). The CD8+ T cell epitopes from NS5B, [NS5B3273-3281, NS5B3434-3442, and NS5B3673-3681],recalled high numbers of IFN-γ+CD8+ T cells in most of the BVDV-immunized steers (FIG. 5), and were broadly reactive against BVDV-1 and -2 strains (Table 3).

### Novel BVDV CD8+ T Cell Epitopes are Bovine MHC I-Restricted

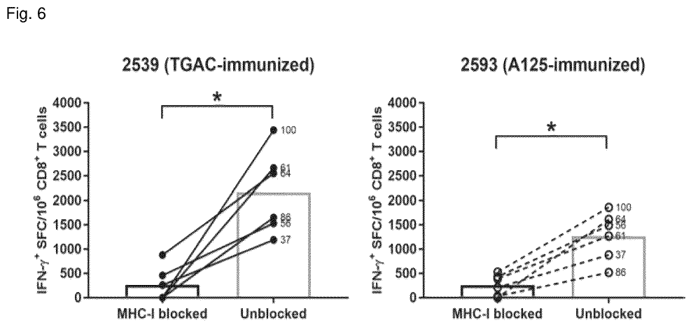

IFN-γ+CD8+ T cells were consistently recalled by Npro95-103, Erns493-501, E1610-618, E2999-1007, NS4B2585-2593, and NS5A2783-2791 [Peptides 61, 86, 56, 100, 37, and 64 respectively (Table 3)], in a TGAC (2539) and an A125 vaccinee (2593) (FIG. 6). However, the recall responses by the six BVDV CD8+ T cell epitopes in the presence of anti-bovine MHC I mAbs were significantly reduced (*p<0.05) (FIG. 6). The inhibition of epitope-specific CD8+ T cell recall responses in BVDV-immunized steers due to MHC I blockade therefore confirmed that the novel defined BVDV epitopes are bovine MHC I-restricted.

### Discussion

Although the presence of BVDV-specific CD8+ T cells in the vaccinated and infected cattle have been documented, identification of CD8+ T cell epitopes and evaluation of their importance for mediating protective immunity against BVDV is not well studied. Previously defined BVDV neutralizing epitopes [from E2] and MHC-DR-restricted CD4+ T cell epitopes [from E2 and NS3] were recently used to generate a rationally designed BVDV subunit vaccine which conferred significantly better cross-protection in cattle than the traditional MLV and KV vaccines. Unlike the hypervariable neutralizing B cell epitopes, Flavivirus-specific CD8+ T cell epitopes, from both structural and nonstructural antigens, tend to be highly conserved and therefore, are broadly reactive against heterologous strains. To increase vaccine coverage and efficacy, discovery of novel BVDV CD8+ T cell determinants is paramount. Hence with that goal in mind, we integrated in silico epitope prediction (NetMHCpan2.8) with the ex vivo validation of the predicted epitopes using outbred steers to identify defined BVDV CD8+ T cell epitopes. Steers were infected with BVDV-1 and then boosted multiple times with gamma-irradiated BVDV-1 or -2 to facilitate MHC I-restricted presentation of BVDV antigens which subsequently, primed, and expanded BVDV-specific CD8+ T cells. For the first time, BVDV-specific CD8+ T cells elicited in steers were demonstrated in the present study and were shown to be highly cross-reactive. The CD8+ T cells from these steers were then employed to screen pools of predicted bovine MHC I-binding peptides that recalled high levels of IFN-γ-secreting CD8+ T cell responses.

Individual peptides from the positive pools were analyzed for recalling IFN-γ+CD8+ T cell responses in BVDV-1- and -2-immunized steers in order to determine the extent of cross-reactivity in the responding CD8+ T cell repertoires (data not shown). Several predicted peptides from the positive pools were identified as strong IFN-γ-inducers that are highly conserved and are located within BVDV structural and nonstructural antigens. Erns, which helps BVDV in establishing persistent infection by its RNase activity, elicits BVDV-specific T cell responses, but defined T cell epitopes from Erns have not been reported. In this study, defined IFN-γ-inducing CD8+ T cell epitopes that are conserved across BVDV-1 and -2, were identified from Erns (Erns363-371, Erns488-496, Erns493-501, and Erns496-504). E1 and E2 heterodimers form the outer envelope of BVDV. While E2 is a protective antigen against BVDV, E1 has not been studied for its contribution to protective immunity. Three IFN-γ-inducing CD8+ T cell epitopes were identified within E1. Remarkably, two E1-derived epitopes (E1552-560 and E1628-636) which are present only in BVDV-1 strains, induced IFN-γ responses in CD8+ T cells from BVDV-2-immunized steers. Since the immunized steers had previously recovered from a BVDV-1 infection, these responses observed in BVDV-2-immunized steers indicate that the two epitopes are likely immunodominant and have the potential to prime strong memory CD8+ T cells against BVDV-1 strains. Flavivirus E2 antigen contains CD8+ T cell epitopes that induce T cell responses against heterogeneous viruses. In Classical Swine Fever Virus [CSFV], E2 is one of the major CTL targets. In the current study, one potent IFN-γ-inducing CD8+ T cell epitope was discovered from E2 (E2999-1007). In all likelihood, this sole E2-derived epitope, which is highly conserved in more than 200 BVDV strains, could be a broadly protective CTL determinant and therefore, it needs to be further investigated along with its cognate BoLA I haplotype(s).

Other than Erns, Npro, the first non-structural antigen encoded by the viral genome, is another BVDV antigen responsible for causing immunosuppression and persistent infection. While Npro is an important CD4+ T cell target, it is not known whether it elicits CD8+ T cell response during BVDV infection. Two novel CD8+ T cell epitopes predicted from Npro (Npro95-103 and Npro106-114) were shown to be inducers of strong cross-reactive IFN-γ response. BVDV NS2/3 antigens are also targets for CD4+ T cells and are often included in experimental subunit vaccines. Subunit vaccine comprising only of NS3, protects BVDV-infected cattle by alleviating viral burden. Clearly, apart from BVDV-specific CD4+ T cells, NS3 also stimulates CD8+ T cell responses which help in eliminating BVDV-infected cells. Moreover, NS2/3-derived CTL epitopes have been identified in CSFV and in other Flaviviruses. From BVDV NS2, two CD8+ T cell epitopes (NS21195-1203- and NS21407-1415) that are conserved in 95 BVDV-1a and -1b strains, were identified. Most notably, broadly reactive CD8+ T cell epitopes, conserved among more than 200 BVDV-1 and -2 strains, were discovered to have originated from NS2/3 (NS21291-1299, NS21373-1381, and NS32010-2018).

The significance of broadly reactive T cell responses mounted by the nonstructural antigens [NS2, NS3, NS4A-B, and NS5A-B], which constitute about 75% of the viral polyprotein, have been emphasized and utilized for designing T cell-based vaccines against key global pathogens that are notorious for their heterogeneity. In addition to sNS2/3, multiple BVDV cross-reactive CD8+ T cell epitopes from NS4 (NS4A2291-2299, NS4B2555-2563, NS4B2568-2576, NS4B2585-2593, NS4B2620-2628, and NS4B2664-2672) and NS5 (NS5A2783-2791, NS5A2992-2930, NS5A3038-3046, NS5B3273-3281, NS5B3434-3442, and NS5B3673-3681) were identified and these are conserved among 178 strains from BVDV-1 and -2 genotypes. However, there was one IFN-γ-inducing CD8+ T cell epitope from NS5A (NS5A3067-3075) which is only present in BVDV-1 genotype.

The results presented here are unique, especially in the context of BVDV and Flaviviruses, since this study sought to identify novel CD8+ T cell epitopes from various regions of the BVDV polyprotein. The outcome also corroborates that high frequencies of long-term BVDV-specific memory CD8+ T cells created during infection are localized in the spleen. This was made apparent by the consistent recall responses detected in the BVDV-2-immunized steers, which had undergone BVDV-1 infection prior to the immunization, against the epitopes that were conserved only in BVDV-1. Undeniably, within the BVDV polyprotein, there are numerous conserved as well as sub-genotype-specific T cell epitopes that can impart long-term protective T cell immunity and thereby, mitigate BVDV infection prevalence in herds. Hence, BVDV vaccination strategy should aim to incorporate divergent and conserved T cell epitopes for protection against diverse circulating BVDV strains. Furthermore, comprehensive assessment of IFN-γ-inducing CD8+ T cell epitopes will certainly yield novel protective determinants which will reshape the landscape of BVDV vaccine immunology and advance the BVDV eradication programs.

### EXAMPLE 2

This example generates a BPI3Vc backbone for use as a vector and for delivery and/or expression of antigens in an animal in need thereof.

The BPI3V Genotype C strain TVMDL16 was used as a vaccine strain and vector expressing BVDV E2 antigen.

Fully sequenced complete BPI3V genomes in the US were retrieved from NCBI and aligned. They cluster into 3 main clades representing Genotype A, B, and C, which is consistent with previous reports. The NC 002161.1 BPI3V complete genome, AF 178654.1 BPI3V strain Kansas/15626/84 complete genome, AF 178655.1 BPI3V Shipping Fever complete genome, KJ647288.1 BPI3V isolate TVMDL24 complete genome, and KJ647289.1 BPI3V isolate TVMDL60 complete genome were identified as belong to genotype A. The KJ647284.1 BPI3V isolate TVMDL15 complete genome, KP764763.1 BPI3V strain TtPIV-1 complete genome, and KJ647286.1 BPI3V isolate TVMDL17 complete genome were identified as belonging to genotype B. The KJ647285.1 BPI3V isolate TVMDL16 complete genome and KJ647287.1 BPI3V isolate TVMDL20 complete genome were identified as belonging to genotype C.

The BPI3V Genotypes C TVMDL16 and TVMDL20, protein and nucleotide sequences for the following BPI3V Genotype C isolates in different parts of the world were aligned as shown in Table 4.

TABLE 4

| Virus isolate | Accession # | Country |
|---|---|---|
| Bovine parainfluenza virus 3 isolate | KJ647285.1 | USA |
| TVMDL16, complete genome | | |
| Bovine parainfluenza virus 3 isolate | KJ647287.1 | USA |
| TVMDL20, complete genome | | |
| Bovine parainfluenza virus 3 strain SD0835, | HQ530153.1 | China |
| complete genome | | |
| Bovine parainfluenza virus 3 isolate 12Q061, | JX969001.1 | S. Korea |
| complete genome | | |
| Bovine parainfluenza virus 3 strain NX49, | KT071671.1 | China |
| complete genome | | |
| Bovine parainfluenza virus 3 viral cRNA, | LC000638.1 | Japan |
| complete genome, isolate: HS9 | | |

Following alignment, regions where an amino acid from the TVMDL16 strain differed from the TVMDL20 strain were identified. This particular site was compared to the other four aligned sequences to determine the most dominant consensus as exemplified below in Table 5 for the phosphoprotein.

### Amino Acid Alignment

TABLE 5
with SEQ ID NOS. 201-224, respectively.

| Domain Data | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

Nucleotide alignment is shown below in Table 6 which includes SEQ ID NOS. 225-248, respectively.

TABLE 6

| | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

Based on these alignments, the total number of TVMDL16 or TVMDL20 variable sites that were similar to the rest aligned sequences were added and results obtained as shown in Table 7 below:

TABLE 7

| Protein/Nucleotide | TVMDL16 | TVMDL20 |
|---|---|---|
| Nucleoprotein | 0 | 2 |
| Phosphoprotein | 12 | 2 |
| Matrix | 0 | 0 |
| Fusion | 0 | 1 |
| Hemagglutinin-neuraminidase | 3 | 1 |
| Large polymerase | 7 | 5 |
| Total | 22 | 11 |
| Nucleotide genome Total | 104 | 73 |

### Attenuating BPIV-3 TVMDL16 (Mutation “a”)

Attenuation based on mutations obtained from current vaccine strains: The selected BPI3Vc TVMDL16 genome was aligned together with BPI3V vaccine strains in use in the US, Shipping Fever strain and Kansas/15626/84, which are both Genotype A. Other published Genotypes A and C sequences were also included in order to identify specific sites which are conserved only for the US vaccine strains. Table 8 shows the genomes used in this alignment and Table 9 provides the alignment of SEQ ID NOS. 249-276, respectively.

TABLE 8

| Virus isolate | Accession # | Country | Genotype |
|---|---|---|---|
| Bovine parainfluenza virus 3 strain | JQ063064.1 | China | A |
| NM09 from China, complete | | | |
| genome | | | |
| Bovine parainfluenza virus 3 DNA, | D84095.1 | Japan | A |
| complete genome | | | |
| Bovine parainfluenza virus 3 strain | AF178655.1 | US | A |
| Shipping Fever, complete genome | | | |
| Bovine parainfluenza virus 3 | KJ647288.1 | US | A |
| isolate TVMDL24, complete | | | |
| genome | | | |
| Bovine parainfluenza virus 3 | KJ647289.1 | US | A |
| isolate TVMDL60, complete | | | |
| genome | | | |
| Bovine parainfluenza virus 3 viral | AB770484.1 | Japan | A |
| cRNA, complete genome, strain: | | | |
| BN-1 | | | |
| Bovine parainfluenza virus 3 viral | AB770485.1 | Japan | A |
| cRNA, complete genome, strain: | | | |
| BN-CE | | | |
| Bovine parainfluenza virus 3 strain | AF178654.1 | US | A |
| Kansas/15626/84, complete | | | |
| genome | | | |
| Bovine parainfluenza virus 3 | KJ647285.1 | USA | C |
| isolate TVMDL16, complete | | | |
| genome | | | |
| Bovine parainfluenza virus 3 | KJ647287.1 | USA | C |
| isolate TVMDL20, complete | | | |
| genome | | | |
| Bovine parainfluenza virus 3 strain | HQ530153.1 | China | C |
| SD0835, complete genome | | | |
| Bovine parainfluenza virus 3 | JX969001.1 | S. Korea | C |
| isolate 12Q061, complete genome | | | |
| Bovine parainfluenza virus 3 strain | KT071671.1 | China | C |
| NX49, complete genome | | | |
| Bovine parainfluenza virus 3 viral | LC000638.1 | Japan | C |
| cRNA, complete genome, isolate: | | | |
| HS9 | | | |

Specific amino acids variable only for the vaccine strains in US but conserved across the Genotypes A and C were identified below in Table 9.

TABLE 5

| | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

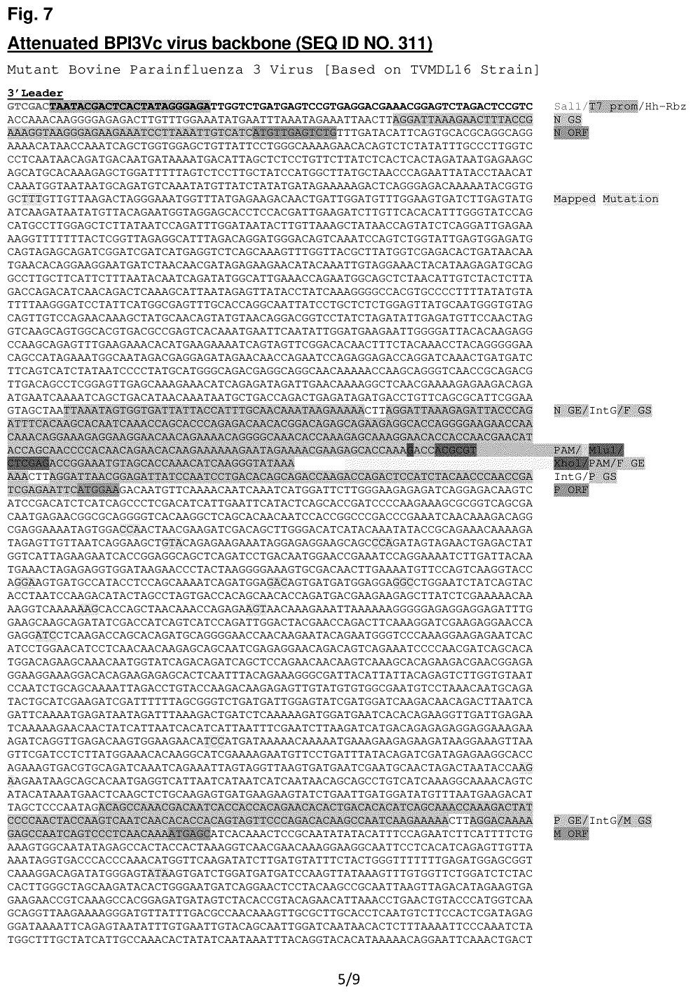

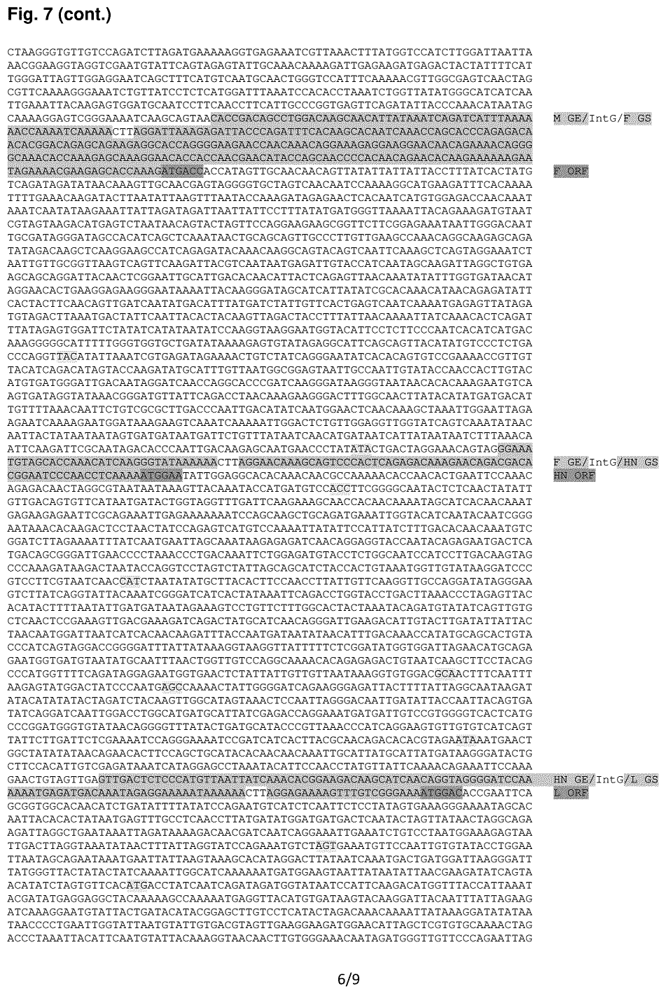

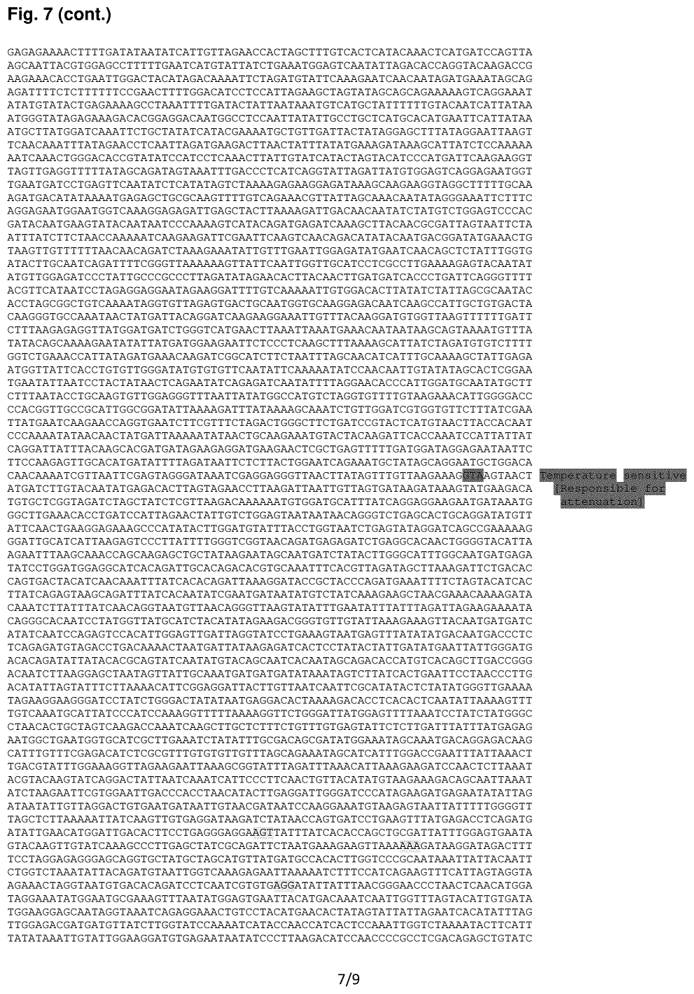

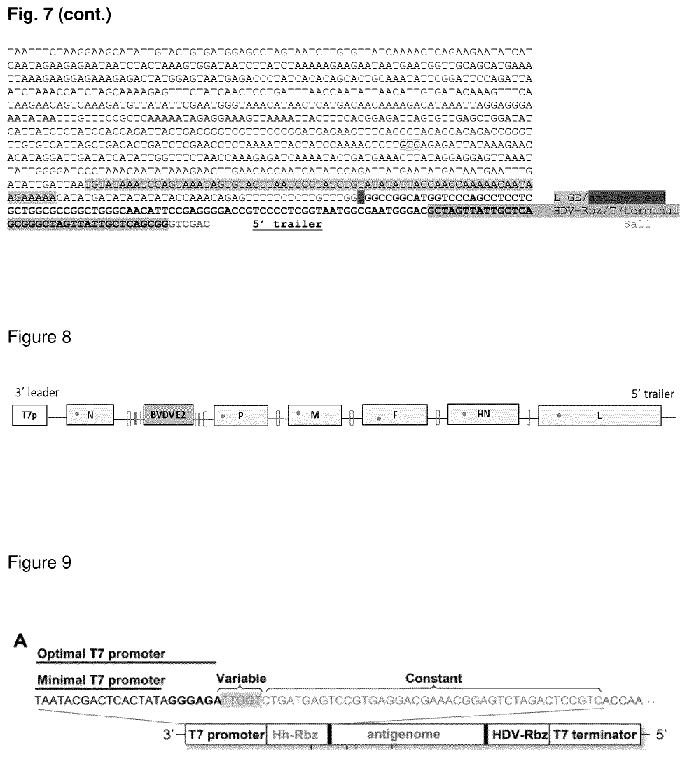

A sample of these sites that formed the basis of creating exact mutations on the TVMDL16 strain to create a mutant BPI3V TVMDL16 is shown below in Table 10, which includes SEQ ID NOS. 277-291, respectively. The complete mutated sequence is provided in the sequence listing as SEQ ID NO. 292 and is shown in FIG. 7.

TABLE 10

| | |
|---|---|
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

Temperature sensitive attenuating mutation (mutation “b”). A distinct temperature sensitive single substitution in the polymerase gene, I 1103 V (change from Isoleucine to Valine in position 1103) was previously identified to cause temperature sensitive (ts) and attenuated phenotype in the reference Kansas/15626/84 vaccine strain. In this regard, this substitution was also made in some forms of the mutant BPI3V TVMDL16 genome at position 1103 from Isoleucine (ATA) to Valine (GTA).

The combination of mutation a and mutation b form a preferred form of the BPI3Vc vector platform shown below in Table 11, which includes SEQ ID NOS. 293-305, respectively.

TABLE 11

| | |
|---|---|
| [symbol] | [symbol] |
| [symbol] | [symbol] |
| [symbol] | [symbol] |
| [symbol] | [symbol] |
| [symbol] | [symbol] [symbol] |
| [symbol] | [symbol] |
| [symbol] | [symbol] [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |
| [symbol] | [symbol] |

[symbol]

indicates data missing or illegible when filed

The above mentioned modifications (a and b) created the ‘Mutant BPI3V TVMDL16 genome.

### Design of a Vaccine Vector from Mutant BPIV3 TVMDL16 Genome

Insert position and design: BPI3Va has previously been used as a vaccine vector for expressing foreign proteins of Human parainfluenza virus-3 and Respiratory syncytial virus, while being able to retain its infectivity and immunogenicity. The position of insertion in the parainfluenza virus genome determines the level of expression of gene of interest. Higher levels of expression are observed with inserts placed at closer to the 3′ end of the negative sense genome and level of expression decreases with downstream insert positions.

Mutant BPI3V TVMDL16 was therefore designed for the insertion to be placed closer to 3′ end of the genome, immediately downstream of the Nucleoprotein as illustrated in FIG. 8. As can be seen in FIG. 8, which provides the design of a BPI3Vc-E2^{b} backbone, the BVDV E2^{b} transgene is located between N and P, which has been shown to be suitable transgene insertion site for generation of recombinant BPI3V constructs including those of the present disclosure (SEQ ID NOS. 1-200). The green dots indicate location of attenuating mutations based on the current BPI3Va vaccine virus strain [Kansas/15626/84]. Specifically, I 1103 V mutation in the polymerase gene (L) is responsible for temperature sensitive [Ts mutant] attenuation.

With the intention of expressing the insertion sequence on the surface of the virus, the idea will be to mimic the assembly of the BPI3V Fusion protein. Hence a Fusion (F) gene start sequence, transmembrane, cytoplasmic domains flank the insertion sequence. PAM sites for possible exploration with CRISPR and restriction sites are placed as shown above in order to allow insertion of target genes

### Optimized T7 Expression Promoter

Reverse genetics system for rescue of negative stranded RNA Paramyxoviruses from plasmids employs the bacteriophage T7 RNA polymerase. This can be obtained in three ways (i) co-infecting cells with vaccinia virus expressing T7, transfecting cell lines that constitutively co-express T7, or (iii) co-transfecting cells with a plasmid expressing T7 polymerase. Rescue efficiency was demonstrated to be significantly increased by use of a T7 polymerase gene codon optimized for expression in mammalian cells (BSR-T7/5 cells) which also constitutively express T7 polymerase. In this case, the promoter sequence in the vector backbone is also respectively codon optimized in line with the optimized polymerase gene. Additionally, an autocatalytic hammerhead ribozyme sequence (Hh-Rbz) introduced downstream of the Optimal T7 promoter self-cleaves immediately before the start of the antigenome therefore ensuring that the rule of six is adhered to. The variable region at the start of the Hh-Rbz is the reverse complement of the start of the antigenome, while the constant region is fixed. The BPI3Vc vector was modified to have similar Optimal T7 promoter and Hh-Rbz as shown in the figure below. We also obtained the Optimized T7 polymerase gene in pCAGGSS (Plasmid #65974) deposited to Addgene by the authors and as shown in FIG. 9.

The entire modified BPI3Vc vector containing a codon-optimized gene encoding BVDV-Ib E2 mosaic antigen fused in-frame to FLAG tag was synthesized and cloned into pUC-SP (outsourced from Bio Basic, Canada) to generate a construct designated pUCBPI3Vc-E2^{b} (insert sequence). Upon receipt of the synthesized product and conducting QC by restriction digest, the pUCBPI3Vc-E2^{b} (insert sequence) was then used as a template to PCR virus rescue helper genes: i.e. the N gene, P gene, and L gene.

### Cloning of Helper Plasmids

Primers were designed to PCR the N, P, and L genes from the pUCBPI3Vc-E2^{b} (insert sequence) construct and similar primers will be designed for constructs expressing at least one of SEQ ID NOS. 1-200. The Optimized T7 promoter region was included in the primer design in order to clone the genes in a suitable cloning vector and be able to increase the expression efficiency in the BSR-T7/5 cells while using the Optimized T7 polymerase gene. Using the same format for the codon optimized T7 expression of the vector, the variable region of each helper plasmid was designed according to its respective reverse complement of the start of its respective antigenome.

BPI3V N Fwd
(SEQ ID NO. 305)
5′ GCGTCGACTAATACGACTCACTATAGGGAGAAACATCTGATGAGTC
CGTGAGGACGAAACGGAGTCTAGACTCCGTCATGTTGAGTCTGTTTGAT
ACATTCAGTGCACGCA 3'
BPI3V N Rev
(SEQ ID NO. 306)
5' GCAAGCTTTTAGCTACTTCCGAATGCGCTGAACAGGTC 3'
BPI3V P Fwd
(SEQ ID NO. 307)
5' GCGTCGACTAATACGACTCACTATAGGGAGATCCATCTGATGAGTC
CGTGAGGACGAAACGGAGTCTAGACTCCGTCATGGAAGACAATGTTCAA
AACAATCAAATCATGG 3'
BPI3V P Rev
(SEQ ID NO. 308)
5' GCAAGCTTCTATTGGGAGCTAATGTCTTCATTAAACATATCCATCA
ATTCAGATACTTCT 3'
BPI3V L Fwd-1
(SEQ ID NO. 309)
5' GCCCCGGGTAATACGACTCACTATAGGGAGATCCATCTGATGAGTCC
GTGAGGACGAAACGGAGTCTAGACTCCGTCATGGACACCGAATTCAGC
GGTGGC 3'
BPI3V L Rev
(SEQ ID NO. 310)
5' GCAAGCTTTTAATCAATATCAAATTCATTATCATATTCATAATCTG
GATATGATTGGTGT 3'

PCR amplified N, P, and L genes were cloned into pCR4-TOPO vector and QC by restriction digest and sequencing.

### Recombinant BPI3Vc-E2^{b} (Insert Sequence) Virus Rescue and Amplification

### Virus Rescue and Amplification

Seed BSR-T7/5 cells at 4×10⁵ per well in a 6-well plate in order to achieve ˜50% confluence on the next day of transfection.

Transfection constructs: Use the following amounts of N, P, and L helper plasmid constructs, and a plasmid encoding T7 polymerase:
5 μg pUCBPI3Vc-E2^{b} (insert sequence) construct
1.5 μg N construct
0.8 μg P construct
0.1 μg L construct
5 μg of T7 polymerase construct

### Transfection Reagents

Set up 1:
5.5 μl PLUS reagent
9 μl Lipofectamine LTX
200 μl Opti-MEM
Set up 2:
2.5 μl PEI per microgram of DNA
200 μl Opti-MEM

Add the transfection reagents (PEI or Lipofectamine/PLUS reagent diluted in 25 μl Optimem) to the plasmid constructs (diluted in 25 μl Optimem) and mix by pipetting gently.

Transfer the constructs/transfection reagents to 150 μl of Optimem.

Incubate at room temperature for 30 min.

Add the transfection mixture gently onto the cells (It is critical that mixture not be agitated before adding to cells, as mixing can disrupt the liposomes at this point).

Incubate at 37° C. for 72 hours (3 days).

At 72 hours post-transfection, harvest the P (0) media and cells and freeze-thaw only the cells (one cycle). Spin down and mix the clean supernatant. Use this to infect fresh MDBK cell monolayer. Stain a portion of the 6 well plate with anti-Flag/E2-specific mAb or sera/anti-BPI3V reference serum to confirm virus assembly.

Infect fresh monolayer of MDBK cells in a T25 flask with the lysate to generate P1 virus stock. Incubate at 37° C. for 5 days.

Stain a portion of the T25 flask as aboveto confirm virus replication.

Harvest the P (1) virus stock as above and infect fresh monolayer of MDBK cells in a T75 flask.

Incubate at 37° C. for 5 days, harvest P (2) virus and infect a 6well MDBK plate for 3 days (72 hours) for staining as shown below;

Stain the 6 well plate with:
Anti-Flag antibody—confirm insert sequence expression.
Anti-BPI3V IgG polyclonal antibody—confirm that the virus assembled is BPI3V.

Anti-E2 monoclonal antibody—confirm E2 protein insert sequence expression.

Amplify virus in T7 then T175 flasks and conduct confirmatory QC at each time point QC (by staining as above). Purify virus by sucrose gradient and determine virus titer. Conduct QC of the purified virus and conduct in vivo studies to determine attenuation and vaccine efficacy.

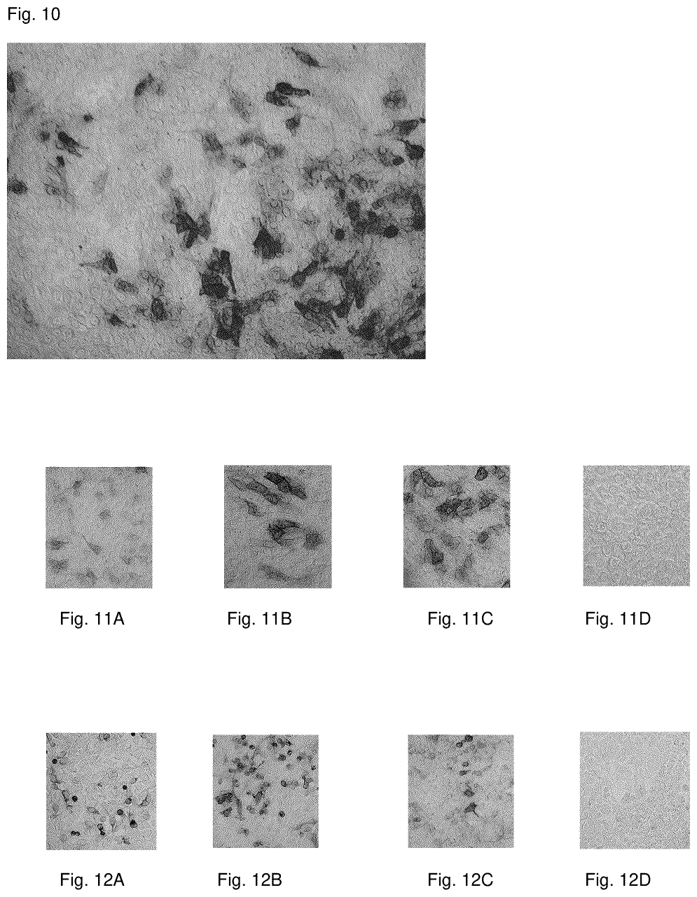

FIG. 10 illustrates an attenuated BPI3Vc-E2^{b} virus expressing the E2^{b} transgene. For this figure, the recombinant BPI3Vc virus expressing the FLAG-tagged E2^{b} transgene was rescued by transfecting BSR-T7/5 cells, which constitutively express the T7 RNA polymerase with the pBPI3Vc-E2^{b} construct in the presence of the pCR4-N, pCR4-P, and pCR4-L helper constructs. Lysate and supernatant from the transfected cells was used to infect MDBK and 72 hrs. post-infection, expression of the FLAG-tagged E2^{b} was evaluated by immunocytometric analysis using anti-FLAG monoclonal antibody. The rescued virus can be scaled up, and tested for attenuation in vitro and in vivo. It can also be used to conduct a pilot immunogenicity and protective efficacy against BPI3V genotype C strains.

FIGS. 11A-11D are photographs illustrating the surface display of a BVDV E2^{b} transgene on cells infected with BPI3Vc-E2^{b} virus. Rescued recombinant BPI3Vc-E2^{b} virus was used to infect MDBK cells and at 72 hours post-infection, immunocytometric analysis of unfixed cells was used to validate expression of BVDV E2^{b} transgene by BPI3Vc-E2^{b} virus on cell surface using (FIG. 11A) anti-FLAG monoclonal antibody; (FIG. 11B) BPI3V polyclonal antibody (detects expression of BPI3Vc antigens); (FIG. 11C) BVDV Type 1&2 monoclonal antibody (mAb 348) against E2; and (FIG. 11D) uninfected negative control. This is QC data shows that the BVDV E2^{b} transgene is expressed on the surface of cells infected with the BPI3Vc-E2^{b} virus [11A, 11C]. The data [11B] also shows that the rescued virus is strongly recognized by BPI3V reference serum (APHIS 475 BDV 0601).

FIGS. 12A-D are photographs illustrating the authenticity of mosaic BPI3V F2-HN2 expressed by plasmid constructs. The expression and authenticity of FLAG-tagged mosaic novel fusion [F2] and HIS-tagged Hemagglutinin-Neuraminidase [HN2] proteins was evaluated by immunocytometric analysis of HEK-293A cells transfected with plasmid constructs and probed with (FIG. 12A) Anti-FLAG monoclonal antibody to detect the FLAG-tagged Fusion protein; (FIG. 12B) Anti-HIS monoclonal antibody to detect the HIS-tagged Hemagglutinin-Neuraminidase protein; (FIG. 12C) BPI3V polyclonal antibody and (FIG. 12D) pCDNA construct negative control. In some forms, the F2-HN2 open-reading frame is separated by a 2A autocleavable motif to allow generation of the individual F2 and HN2 antigens. This data shows expression of the novel mosaic F2HN2 antigens. Antigen expression was validated using anti-tag mAbs and then authenticated using the BPI3V reference serum mentioned above.

What is claimed is:

1. An immunogenic composition comprising:
an antigenic CD8+ T cell epitope derived from at least one bovine viral diarrhea virus (BVDV) antigen selected from the group consisting of N^{pro}, E^{rns}, E1, E2, NS2-3, NS4A-B, NS5A-B, and any combination thereof; and
a pharmaceutical or veterinary acceptable carrier selected from the group consisting of a solvent, a dispersion media, a coating, a stabilizing agent, a preservative, an antimicrobial agent, an antifungal agent, an isotonic agent, and an adsorption delaying agent, and any combination thereof.

2. The immunogenic composition of claim 1, wherein the at least one BVDV antigen is a sequence having at least 85% sequence identity with a sequence selected from the group consisting of SEQ ID NOS. 1-200.

3. The immunogenic composition of claim 1, wherein the at least one BVDV antigen is a sequence having at least 85% sequence identity with a sequence selected from the group consisting of 61, 45, 176, 88, 86, 47, 32, 56, 34, 100, 39, 97, 82, 69, 87, 177, 172, 63, 37, 99, 43, 64, 65, 81, 40, 38, 89, 173, or any combination thereof.

4. The immunogenic composition of claim 1, wherein the immunogenic composition comprises at least 2 CD8+ T cell antigenic epitopes.

5. The immunogenic composition of claim 1, wherein the BVDV is a BVDV-1 or BVDV-2 genotype.

6. The immunogenic composition of claim 1, further comprising an adjuvant.

7. The immunogenic composition of claim 1, wherein the BVDV antigen is expressed by a vector.

8. The immunogenic composition of claim 7, wherein the vector is derived from Bovine Parainfluenza Type 3 c virus.

9. The immunogenic composition of claim 1, wherein the immunogenic composition is effective at reducing the severity or incidence of clinical signs of BVDV-1a, BVDV-1b, and BVDV-2.

10. A method of reducing the incidence or severity of clinical signs caused by BVDV comprising the step of administering the composition of claim 1 or claim 16 to an animal in need thereof.

11. The method of claim 10, wherein the incidence or severity of clinical signs are reduced at least 10% in comparison to an animal or group of animals that have not received an administration of the composition of claim 1.

12. The method of claim 10, wherein the composition of claim 1 is administered multiple times to the animal in need thereof.

13. The method of claim 10, wherein the clinical signs are selected from the group consisting of bloody diarrhea, high fever (105-107 degrees), off-feed, mouth ulcers, pneumonia, reduced weight gain, abortion, and the birth of persistently infected (PI) carrier calves that shed infectious BVDV.

14. The method of claim 10, wherein the BVDV is selected from the group consisting of BVDV-1a, BVDV-1b, and BVDV-2.

15. The method of claim 10, wherein the clinical signs are caused by at least two of BVDV-1a, BVDV-1b, and BVDV-2.

16. An immunogenic composition comprising:
a BPI3Vc vector expressing at least an antigenic CD8+ T cell epitope derived from at least one bovine viral diarrhea virus (BVDV) antigen selected from the group consisting of N^{pro}, E^{rns}, E1, E2, NS2-3, NS4A-B, NS5A-B, and any combination thereof; and
a pharmaceutical or veterinary acceptable carrier selected from the group consisting of a solvent, a dispersion media, a coating, a stabilizing agent, a preservative, an antimicrobrial agent, an antifungal agent, an isotonic agent, and an adsorption delaying agent, and any combination thereof.

17. The immunogenic composition of claim 16, wherein the BVDV antigen is a sequence having at least 85% sequence identity with a sequence selected from the group consisting of SEQ ID NOS. 1-200.

18. The immunogenic composition of claim 16, wherein the BVDV antigen is a sequence having at least 85% sequence identity with a sequence selected from the group consisting of 61, 45, 176, 88, 86, 47, 32, 56, 34, 100, 39, 97, 82, 69, 87, 177, 172, 63, 37, 99, 43, 64, 65, 81, 40, 38, 89, 173, or any combination thereof.

19. The immunogenic composition of claim 16, wherein the immunogenic composition comprises at least 2 antigenic epitopes.

20. The immunogenic composition of claim 16, further comprising an adjuvant.

21. The immunogenic composition of claim 1, wherein the immunogenic composition is effective at reducing the severity or incidence of clinical signs of BVDV-1a, BVDV-1b, and BVDV-2.
